(19) United States
(12) Patent Application Publication
(10) Pub. No.: US 2016/0193059 A1
(43) Pub. Date: Jul. 7, 2016

(54) INTRALUMINAL IMPLANTS AND METHODS

(71) Applicant: APERIAM MEDICAL, INC., Roise, ID (US)

(72) Inventors: Alexander Joseph Asconeguy, Murrieta, CA (US); Gerald Martin Stobbs, Alpine, CA (US); Michael William Craven, Eden Prairie, MN (US); Karmi Nicole Robison, Vista, CA (US); Jeffery James Edwards, Austin, TX (US); Steven Wayne Bence, Vista, CA (US)

(73) Assignee: Aperiam Medical, lnc., Boise, ID (US)

(21) Appl. No.: 14/915,367

(22) PCT Filed: Jul. 21, 2014
(86) PCT No.: PCT/US2014/047366

§ 371 Date: Feb. 29, 2016

Related U.S. Application Data
(60) Provisional application No. 61/856,598, filed on Jul. 19, 2013.

(51) Int. Cl. A61F 2/844; A61F 2/966

(52) U.S. Cl. CPC A61F 2/844; A61F 2/966

(57) ABSTRACT

Devices and methods are described for treating maladies such as atrial fibrillation. The devices and methods, in some implementations, include implant comprising a ribbon or other structure formed into a desired helical or other shape. The ribbon can provide mechanical pressure against an adjacent tissue, e.g., the tissue of a vessel, so as to help at least partially inhibit the propagation of electrical signals along the vessel.

### CROSS-REFERENCE TO RELATED APPLICATIONS

This application claims benefit of priority to U.S. Provisional Patent Application Ser. No. 61/856,598, filed Jul. 19, 2013, incorporated by reference herein. This application is also related to U.S. patent application Ser. No. 13/106,343, filed May 12, 2011 and published as U.S. Publication No. 2011/0282343, U.S. patent application Ser. No. 13/457,033, filed Apr. 26, 2012 and published as U.S. Publication No. 2012/0277842, U.S. patent application Ser. No. 13/655,351, filed Oct. 18, 2012 and published as U.S. Publication No. 2013/0109987 and U.S. patent application Ser. No. 13/830,040, filed Mar. 14, 2013 and published as U.S. Publication No. 2013/0204311, all of which are owned by the assignee of the present application and are incorporated by reference herein in their entirety.

### BACKGROUND

1. Field

This application relates generally to intraluminal (e.g., intravascular) implants configured to at least partially block or interrupt electrical and/or neural signals running adjacent a vessel or other body lumen of a subject, as well as various tools, devices, systems and methods related thereto.

2. Description of the Related Art

Atrial fibrillation and other cardiac arrhythmias are common and dangerous diseases. For example, atrial fibrillation patients have a greatly increased risk of stroke mortality. Atrial fibrillation disrupts the normal sinus rhythm of subjects. For instance, during atrial fibrillation multiple wavefronts circulate rapidly and chaotically through the atria, causing them to contract in an uncoordinated and ineffective manner at increased rates. Symptoms arise from the rapid, irregular pulse as well as the loss of cardiac pump function related to uncoordinated atrial contractions. These uncoordinated contractions also allow blood to pool in the atria and may ultimately lead to thromboembolism and stroke. Other electrical signal or nerve mediated diseases can also be affected by impacting the signals and/or neural activity adjacent a body lumen.

### SUMMARY

According to some embodiments, an implant configured for placement within a body lumen (e.g., artery, vein, other vessel, airway, etc.) of a subject comprises a ribbon having a generally rectangular cross-sectional shape. In some embodiments, the ribbon comprises at least a partially helical, curved or wound shape. In some embodiments, the ribbon comprises a smooth outer surface with no penetrating elements or features. In some embodiments, the implant is configured to be moved between a radially collapsed configuration and a radially deployed configuration, wherein the implant is in the radially collapsed configuration during intraluminal delivery of the implant to the target vessel or other body lumen. In some embodiments, when in the radially deployed configuration, the implant radially expands to contact and exert pressure along adjacent tissue of the target vessel or other body lumen without penetrating said adjacent tissue. In some embodiments, the pressure exerted by the deployed implant at least partially blocks electrical signals along the target vessel or other body lumen. According to some embodiments, deployment of the implant within a target lumen causes the adjacent tissue of the lumen to be stretched in the radial (e.g., circumferential) and/or axial (e.g., longitudinal) directions. The amount of stretching can vary along a length of the target lumen. In some embodiments, the stretching of the adjacent tissue of the lumen causes a tenting effect on the lumen at or near the location of the implant.

According to some embodiments, an implant configured for placement within a target vessel or other body lumen of a subject comprises a ribbon having a generally rectangular cross-sectional shape, wherein the ribbon includes at least a partially helical, wound or curved shape. In some embodiments, the ribbon comprises a smooth outer surface with no penetrating elements or features. In some embodiments, the implant is configured to be moved between a radially collapsed configuration and a radially deployed configuration, wherein the implant is in the radially collapsed configuration during intraluminal delivery of the implant to the target vessel or other body lumen. In some embodiments, when in the radially deployed configuration, the implant radially expands to contact and exert pressure along adjacent tissue of the target vessel or other body lumen and/or stretch (e.g., radially and/or axially) without penetrating the adjacent tissue. In some embodiments, the pressure exerted by the deployed implant at least partially blocks electrical signals along the target vessel or other body lumen. In some embodiments, the ribbon comprises a tail or extension along a first end of the implant, wherein a pitch of adjacent windings of the ribbon along the tail or extension is greater along the first end than a pitch along a second end of the implant such that the implant is generally asymmetrical.

According to some embodiments, the width of the ribbon is about 0.5 mm to 25 mm, e.g., 1 to 10 mm (e.g., about 1 mm, 1-1.1 mm, 1.1-1.2 mm, 1.2-1.3 mm, 1.3-1.4 mm, 1.4-1.5 mm, 1.5-1.6 mm, 1.6-1.7 mm, 1.7-1.8 mm, 1.8-1.9 mm, 1.9-2 mm, 2.1-2.2 mm, 2.2-2.3 mm, 2.3-2.4 mm, 2.4-2.5 mm, 2.5-2.6 mm, 2.6-2.7 mm, 2.7-2.8 mm, 2.8-2.9 mm, 2.9-3 mm, 3-3.1 mm, 3.1-3.2 mm, 3.2-3.3 mm, 3.3-3.4 mm, 3.4-3.5 mm, 3.5-3.6 mm, 3.6-3.7 mm, 3.7-3.8 mm, 3.8-3.9 mm, 3.9-4 mm, 4-4.1 mm, 4.1-4.2 mm, 4.2-4.3 mm, 4.3-4.4 mm, 4.4-4.5 mm, 4.5-4.6 mm, 4.6-4.7 mm, 4.7-4.8 mm, 4.8-4.9 mm, 4.9-5 mm, 5-5.5 mm, 5.5-6 mm, 6-6.5 mm, 6.5-7 mm, 7-7.5 mm, 7.5-8 mm, 8-8.5 mm, 8.5-9 mm, 9-9.5 mm, 9.5-10 mm, values between the foregoing ranges, etc.), e.g., 10-20 mm (e.g., 10-11 mm, 11-12 mm, 12-13 mm, 13-14 mm, 14-15 mm, 15-16 mm, 16-17 mm, 17-18 mm, 18-19 mm, 19-20 mm, 20-21 mm, 21-22 mm, 22-23 mm, 23-24 mm, 24-25 mm, values between the foregoing ranges, etc.) and/or the like. In other embodiments, the width of the ribbon is less that 0.5 mm or greater than 25 mm (e.g., 25-30 mm, 30-35 mm, 35-40 mm, 40-45 mm, 45-50 mm, greater than 50 mm, etc.). According to some embodiments, the thickness of the ribbon is 0.1 mm to 0.5 mm (e.g., 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5 mm, values between the foregoing, etc.). In other embodiments, the thickness of the ribbon is less than about 0.1 mm (e.g., 0.01, 0.02, 0.03, 0.04, 0.05, 0.06, 0.07, 0.08, 0.09, etc.) or greater than 0.5 mm (e.g., 0.5-0.6, 0.6-0.7, 0.7-0.8, 0.8-0.9, 0.9-1, 1-2 mm, more than 2 mm, etc.). Forces delivered and/or pressures exerted by the deployed implant on the target tissue (e.g., a lumen wall) include, but are not limited to, 0.001 N/mm² to 3.5 N/mm² (e.g., between about 0.25 N/mm² and 1.5 N/mm²). In some embodiments, the ribbon of the implant includes sharp or rounded corners or a different overall shape (e.g., rounded, circular or oval profile, along at least a portion of its cross-section). In some embodiments, the ratio of the width to the thickness is 1.5:1 to 30:1 (e.g., approximately 1.5:1, 2:1, 2.5:1, 3:1, 3.5:1, 4:1, 4.5:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 219:1, 30:1, values between the foregoing ranges, etc.). In other embodiments, the ratio of width to thickness of the ribbon or other structure is less than 1.5:1 or greater than 30:1. In some embodiments, the diameter of the deployed (e.g., implanted) implant is between about 1 mm and about 50 mm (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50 mm, diameters between the foregoing values or ranges, etc.). In other embodiments, the diameter of the implant is less than about 1 mm or greater than about 50 mm. In some embodiments, the implant comprises an aspect ratio of between about 0.1:1 to about 10:1. For example, the implant can include an aspect ratio of 0.5:1 to 3:1 (e.g., 0.5:1, 0.6:1, 0.7:1, 0.8:1, 0.9:1, 1:1, 1.1:1, 1.2:1, 1.3:1, 1.4:1, 1.5:1, 1.6:1, 1.7:1, 1.8:1, 1.9:1, 2:1, 2.1:1, 2.2:1, 2.3:1, 2.4:1, 2.5:1, 2.6:1, 2.7:1, 2.8:1, 2.9:1, 3:1, 3.1:1, 3.2:1, 3.3:1, 3.4:1, 3.5:1, 3.6:1, 3.7:1, 3.8:1, 3.9:1, 4:1, 4.1:1, 4.2:1, 4.3:1, 4.4:1, 4.5:1, 4.6:1, 4.7:1, 4.8:1, 4.9:1, 5:1, ratios between the foregoing values, etc.). In other embodiments, the aspect ratio is less about 0.5:1 (e.g., between 0.1:1 and 0.5:1) or greater than about 5:1 (e.g., 5:1-6:1, 6:1-7:1, 7:1-8:1, 8:1-9:1, 9:1-10:1, greater than 10:1, etc.).

According to some embodiments, the ribbon of the implant comprises at least one opening (e.g., slit, slot, cut, pore, recess, window, etc.) that extends at least partially along a length of the implant. The at least one opening can be continuous or discontinuous (e.g., intermittent) along a length of the ribbon. In some embodiments, the at least one opening of the ribbon extends from or near a first end of the ribbon to a second end of the ribbon. In some embodiments, the at least one opening comprises at least one strut or rib extending across at least a portion of the at least one opening.

According to some embodiments, a pitch of adjacent windings of the ribbon is generally constant along an entire length of the implant. In some embodiments, a pitch of adjacent windings of the ribbon varies along at least a portion of a length of the implant. In some embodiments, the implant comprises a tail or extension along a first end of the implant, wherein a pitch of adjacent windings is greater along the first end than a pitch of adjacent windings along a second end of the implant. In some embodiments, a space exists between adjacent windings of the ribbon. In some embodiments, at least two adjacent windings of the ribbon include a space or clearance.

According to some embodiments, the ribbon is symmetrical along a longitudinal axis of the implant. In some embodiments, the ribbon is asymmetrical along a longitudinal axis of the implant. In some embodiments, the ribbon comprises a shape memory material (e.g., Nitinol). In some embodiments, at least one end of the ribbon comprises a hole for securing to a delivery system, wherein the at least one end of the ribbon comprising the hole is rotated related to an immediately adjacent portion of the ribbon to reduce undesirable torque or stresses on the implant during or after implanting. In some embodiments, at least one end of the ribbon is rotated about 90 degrees relative to the immediately adjacent portion of the ribbon. In some embodiments, at least one end of the ribbon comprises a hole for securing to a delivery system, wherein the at least one end of the ribbon comprising the hole comprising a reduced-material neck region. In some embodiments, at least one end of the ribbon comprises a dogbone shape.

According to some embodiments, a method of treating a condition of a subject comprises delivering, at least partially percutaneously, an implant in accordance with the present disclosure to a target vessel or other body lumen (e.g., vein, artery, other blood vessel, airway, urinary or gastroenterological lumen, etc.) of a subject using a delivery system. The method further includes deploying the implant within the target vessel or body lumen of the subject, such that at least a portion of the outer surface of the ribbon contacts and exerts a pressure along the adjacent tissue of the vessel or other body lumen without penetrating the adjacent tissue. In some embodiments, when deployed, the flat and smooth outer surface of the ribbon is generally parallel with the adjacent tissue of the subject. The method additionally comprises withdrawing the delivery system and leaving the implant positioned within the target vessel or other body lumen of the subject. In some embodiments, the pressure exerted by the implanted implant at least partially interrupts (e.g., blocks, interferes, otherwise affects, etc.) signals (e.g., electrical signals) and/or neural activity along the vessel or other body lumen. In some embodiments, the method treats a cardiac arrhythmia (e.g., atrial fibrillation, other types of fibrillations, etc.). In other embodiments, the method treats renal-induced hypertension. In other embodiments, the method treats COPD, asthma or other pulmonary disease or condition. In other embodiments, the method treats a gastroenterological disease or condition (e.g., GERD).

According to some embodiments, the target vessel or other body lumen of the subject comprises at least one of an artery, vein, other blood vessel, an airway, a lumen of the gastroenterological system and a lumen of the urinary tract. In some embodiments, the implant is positioned within a trunk or main lumen (e.g., aorta, trachea, etc.) that comprises one or more upstream or downstream branches. In some embodiments, the method causes the target lumen to stretch in the longitudinal (e.g., axial) and/or radial (e.g., circumferential) directions, either continuously or intermittently at, along or near the portion of the targeted lumen in which the implant is positioned. In some embodiments, the stretching occurs in locations that are not contacted by a portion (e.g., winding, element, etc.) of the implant. In some embodiments, the stretching (e.g., longitudinal and/or radial) occurs between adjacent windings or elements of the ribbon.

### BRIEF DESCRIPTION OF THE DRAWINGS

These and other features, aspects and advantages of the present application are described with reference to drawings of certain embodiments, which are intended to illustrate, but not to limit, the various inventions disclosed herein. It is to be understood that the attached drawings are for the purpose of illustrating concepts and embodiments of the present application and may not be to scale.

FIGS. 1-4 illustrate cross-sectional views of a vessel or other body lumen with one embodiment of an implant positioned therein;

FIG. 5A illustrates one embodiment of an implant positioned within a subject according to one embodiment;

FIG. 5B illustrates one embodiment of an implant positioned within a subject according to one embodiment;

FIG. 5C illustrates one embodiment of a table that includes oversizing percentages for implant of different diameters;

FIGS. 6A and 6B illustrates views of one embodiment of an implant configured for placement within a vessel or other body lumen;

FIGS. 7A and 7B illustrates views of one embodiment of an implant configured for placement within a vessel or other body lumen;

FIGS. 8A and 8B illustrates views of one embodiment of an implant configured for placement within a vessel or other body lumen;

FIGS. 9A and 9B illustrates views of one embodiment of an implant configured for placement within a vessel or other body lumen;

FIGS. 10A and 10B illustrates views of one embodiment of an implant configured for placement within a vessel or other body lumen;

FIGS. 11A and 11B illustrates views of one embodiment of an implant configured for placement within a vessel or other body lumen;

FIGS. 12A and 12B illustrates views of one embodiment of an implant configured for placement within a vessel or other body lumen;

FIG. 12C illustrates one embodiment of a frame used to manufacture the implant of FIGS. 12A and 12B;

FIG. 13A illustrates a perspective view of one embodiment of an implant configured for placement within a vessel or other body lumen;

FIG. 13B illustrates one embodiment of a frame used to manufacture the implant of FIG. 13A;

FIGS. 14A and 14B illustrates views of one embodiment of an implant configured for placement within a vessel or other body lumen;

FIG. 14C illustrates one embodiment of a frame used to manufacture the implant of FIGS. 14A and 14B;

FIGS. 15A-15T illustrate view of various embodiments of an implant configured for placement within a vessel or other body lumen;

FIGS. 16A-16X illustrate view of various embodiments of an implant having a tail or extension portion and configured for placement within a vessel or other body lumen;

FIG. 17 illustrates a view of one embodiment of an implant configured for placement within a vessel or other body lumen;

FIG. 18 illustrates a view of one embodiment of an implant configured for placement within a vessel or other body lumen;

FIG. 19 illustrates a view of one embodiment of an implant configured for placement within a vessel or other body lumen;

FIGS. 20A-20H illustrate view of various embodiments of an implant having a slot or other opening and configured for placement within a vessel or other body lumen;

FIG. 21 illustrates a side view of one embodiment of an implant comprising a hole or other opening along at least one end;

FIG. 22 illustrates a detailed schematic view of an implant ribbon having a rotated configured along at least one of its ends;

FIGS. 23 and 24 illustrate different perspective views of implant embodiments comprising a turned and dogbone end configuration; and

FIG. 25 illustrates one embodiment of an implant positioned within the aorta for renal denervation.

### DETAILED DESCRIPTION

The discussion and the figures illustrated and referenced herein describe various embodiments of an intraluminal (e.g., intravascular) implant, as well as various tools, systems and methods related thereto. A number of these devices and associated treatment methods are particularly well suited to treat atrial fibrillation, other cardiac arrhythmias, renal-induced hypertension, asthma, COPD, gastroenterological diseases and conditions (e.g., GERD), urinary tract conditions and/or the like. However, the various devices, systems, methods and other features of the embodiments disclosed herein may be utilized or applied to treat any other type of disease or condition that is mediated, at least in part, by electrical signals, nerve activity and/or the like.

### Bidirectional Stretching of Targeted Body Lumens

According to some embodiments, the disruption (e.g., stopping, slowing, otherwise impacting, etc.) of electrical signals and/or nerves traveling along a vessel or other body lumen (e.g., a pulmonary vein, a renal artery, an airway, etc.) can be accomplished by stretching of the vessel or lumen using an expandable implant. Any of the implant embodiments disclosed herein or in embodiments described in applications incorporated by reference herein, including but not limited to U.S. patent application Ser. No. 13/830,040, can be configured to provide the necessary stretching to a vessel or other body lumen. The stretching of the vessel or other body lumen can occur in the radial direction and/or the axial (or longitudinal) direction.

Disruption of the electrical and/or nerve conduction can be used in one or more targeted locations. Thus, implants can be selectively positioned within a target vessel or other body lumen (e.g., a pulmonary vein, a renal artery, another type of blood vessel, an airway, a urinary tract vessel, a lumen of the gastroenterological system, etc.), as desired or required for the treatment of a particular disease or condition. For example, for the treatment of cardiac arrhythmias (e.g., atrial fibrillation), one or more implants can be positioned and deployed within or near a pulmonary vein. Accordingly, the pulmonary vein can be stretched (e.g., radially and/or axially or longitudinally) at one or more target locations, depending on the location of the implant (e.g., within the pulmonary vein, at or near the ostium of the vein, etc.).

According to some embodiments, as discussed in greater detail herein, the localized stretching of the vessel or other lumen results from the deployment of the implant within the vessel or lumen, such that the outer diameter of the deployed implant is larger than the inner diameter of the target vessel or lumen (e.g., before the implant is positioned therein). In some embodiments, for example, the deployed implant is approximately 0% to 10% (e.g., 0-1%, 1-2%, 2-3%, 3-4%, 4-5%, 5-6%, 6-7%, 7-8%, 8-9%, 9-10%, percentages between the foregoing ranges, etc.) greater than the inner diameter of the targeted vessel or body lumen into which the implant is positioned and deployed. In other words, the target lumen is circumferentially stretched or enlarged by this percentage after the implant is inserted and expanded therein. As discussed in greater detail herein, the lumen can be stretched in its longitudinal (e.g., axial) direction by the implant, either in lieu or in addition to any circumferential stretching. In other embodiments, the deployed implant is greater than about 10% of the inner diameter of the targeted vessel or body lumen (e.g., 10-12%, 12-14%, 14-16%, 16-18%, 18-20%, 20-25%, 25-30%, 30-35%, 35-40%, 40-45%, 45-50%, 50-60%, 60-70%, 70-80%, 80-90%, 90-100%, greater than 100%, values between the foregoing, etc.).

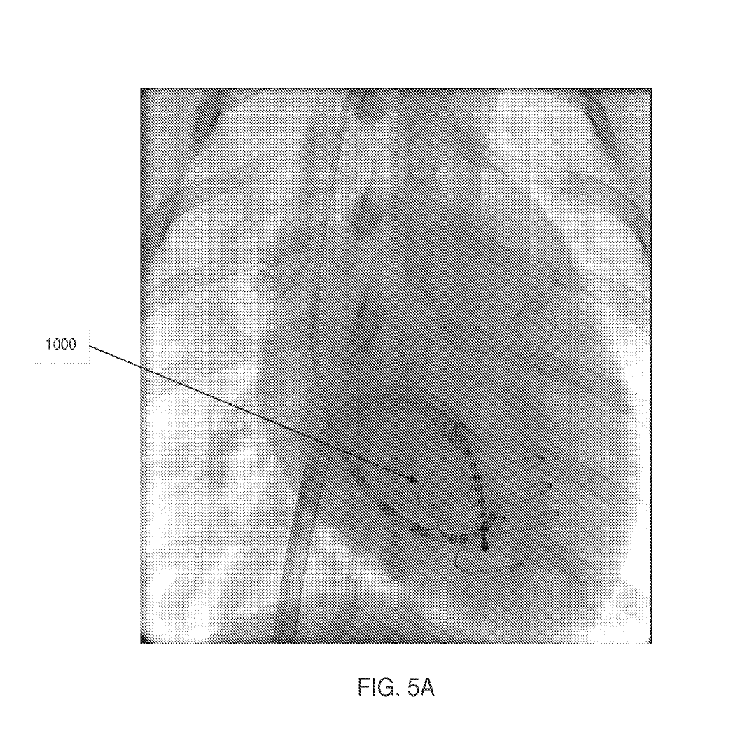

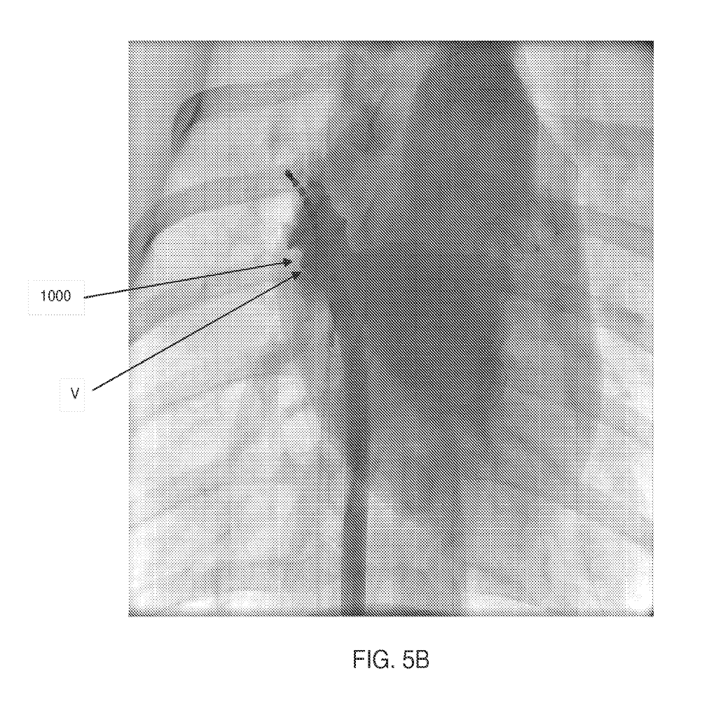

According to some embodiments, the size (e.g., diameter) of the implant is selected based on the actual or approximate size (e.g., diameter) of the target lumen (e.g., vessel, airway, etc.) in which the implant will be positioned. For example, in some embodiments, the deployed diameter of the implant is approximately 0% to 125% (e.g., 0-10%, 10-15%, 15-20%, 20-25%, 25-30%, 30-35%, 35-40%, 40-50%, 50-60%, 60-70%, 70-80%, 80-90%, 90-100%, 100-125%, values between the foregoing ranges, etc.) greater than the diameter (e.g., inner diameter) of the targeted body lumen (e.g., before the implant is deployed therein). In some embodiments, as noted herein, upon deployment within the target lumen, the implant radially expands and exerts a circumferential force or pressure along the adjacent tissue of the lumen (e.g., vein, artery, other vessel, airway, etc.) without penetrating the adjacent tissue. Such a force or pressure can help to at least partially disrupt (e.g., block, interrupt, alter, etc.) electrical signals and/or neural activity along or near the lumen. As noted above, according to some embodiments, one or more portions of the body lumen that are contacted by the implant may radially expand by a particular amount, such as, for example, by about 0% to 100% (e.g., 0-1%, 1-2%, 2-3%, 3-4%, 4-5%, 5-6%, 6-7%, 7-8%, 8-9%, 9-10%, 10-15%, 15-20%, 20-25%, 25-30%, 30-35%, 35-40%, 40-45%, 45-50%, 50-60%, 60-70%, 70-80%, 80-90%, 90-100%, percentages between the foregoing ranges, etc.) relative to the diameter or cross-sectional dimension of the native lumen (e.g., relative to the lumen before the implant is implanted). One embodiment of a table that provides various oversizing percentages for implant of different diameters is provided in FIG. 5C.

In some embodiments, upon deployment within a vessel or other body lumen, an implant can be configured to stretch the targeted vessel or other lumen in both the radial (e.g., circumferential) and axial (e.g., longitudinal) directions. For example, the windings or elements of a helical implant can impart a radial force or stress on the adjacent tissue of the vessel upon expansion of the implant. At the same time, as the windings or elements engage the tissue of the vessel or lumen, an axial or longitudinal stress can occur between adjacent windings or elements. Accordingly, one or more portions of the lumen that are not in direct contact with the implant can be stretched (e.g., longitudinally and/or radially between adjacent windings or elements). As a result, in some embodiments, the lumen (e.g., vessel) can be stretched, in the radial and/or longitudinal directions, both at the locations of the windings or elements or regions between or adjacent the windings or elements. The stretching, both in the radial and the longitudinal directions, helps, in some embodiments, to disrupt (e.g., block, interrupt, alter, etc.) electrical signals and/or neural activity along or near the lumen.

According to some embodiments, the deployed implant can stretch the adjacent vessel or lumen, in the radial (e.g., circumferential) direction only, in the axial (e.g., longitudinal) direction only or in both the radial and the axial directions, by approximately 0% to 10% (e.g., 0-1%, 1-2%, 2-3%, 3-4%, 4-5%, 5-6%, 6-7%, 7-8%, 8-9%, 9-10%) relative to the native condition of the vessel or lumen (e.g., when no implant was positioned therein). In other embodiments, the deployed implant stretches the targeted vessel or body lumen, in the axial and/or radial directions, by more than 10%, (e.g., 10-12%, 12-14%, 14-16%, 16-18%, 18-20%, 20-25%, 25-30%, 30-35%, 35-40%, 40-45%, 45-50%, 50-60%, 60-70%, 70-80%, 80-90%, 90-100%, greater than 100%, values between the foregoing, etc.). In any of the embodiments disclosed herein, the stretching of the lumen in the radial and/or axial directions can be intermittent and/or variable along the portion of the lumen affected by the implant. For example, the portions of the lumen that are contacted by the windings or elements of the implant can experience a greater degree of radial stretching that adjacent portions of the lumen (e.g., the areas between adjacent windings or elements), whereas the areas between adjacent windings or elements of the implant can experience a greater degree of axial stretching (e.g., as a result of the tenting effect caused by the implant).

In some embodiments, by selectively stretching or straining the substrate of the vessel or other body lumen, the implant can promote cellular decoupling. As a result, the ability of myocytes to effectively conduct to an adjacent one by increasing the interstitial spacing between them can be affected. In some embodiments, if the stress imparted by the implant-induced stretching is sufficiently large, an acute initial response occurs, causing disruption of electrical or neural conduction along the vessel or body lumen. In some embodiments, such an initial acute response can be followed by a chronic response, in which fibrogen (e.g., collagen fibers) can fill the interstitial spaces that have been increased due to strain-based myocyte displacement. In turn, in some embodiments, this can create a long-term (e.g., permanent) non-conducting modification of the substrate of the vessel or other body lumen. As noted above, such a stretching (e.g., in the radial and/or axial directions) can be accomplished using any of the implants disclosed herein or in any implants described and discussed in the applications explicitly referenced herein, including but not limited to U.S. patent application Ser. No. 13/830,040.

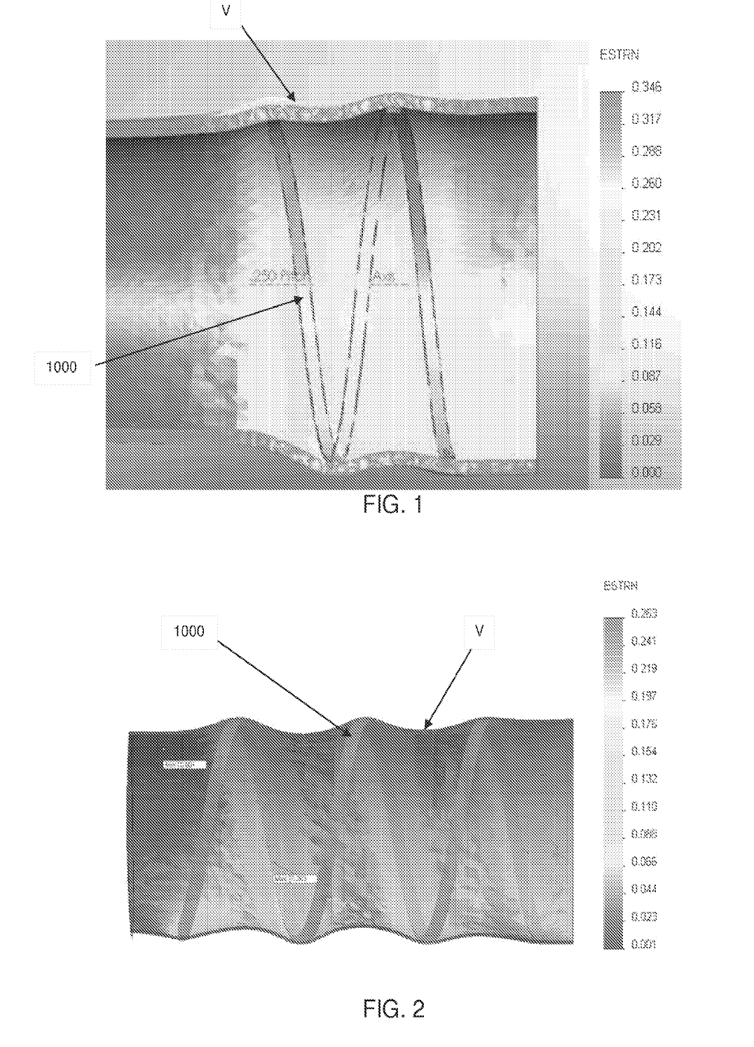

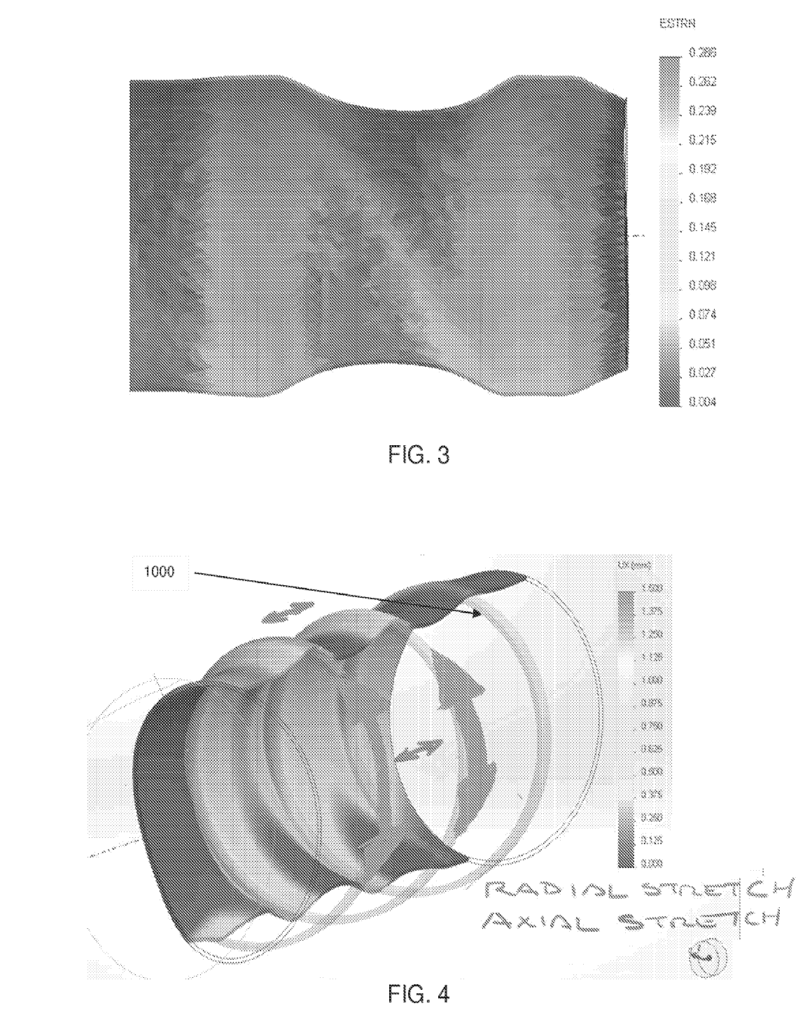

FIGS. 1 to 3 illustrate various embodiments, in cross-sectional view, of finite element analysis (FEA) simulations that model or otherwise predict a deployed implant 1000 within a vessel or other body lumen V (e.g., a pulmonary vein, a renal artery, etc.). FIGS. 1 to 3 denote the location and magnitude of the strain (e.g., stretching) imparted by an implant 1000 on the adjacent tissue of the vessel or lumen V. In some embodiments, certain equivalent strain (ESTRN) values provided in the figures may be multiplied by 100 to be expressed as a percentage. Accordingly, in the embodiment depicted in FIGS. 1 to 3, for example, a vessel can be stretched (e.g., in the radial and/or axial direction) by about 0 to 50%. As shown, depending on the shape of the implant and the orientation of its adjacent windings or elements, the stress imparted on the vessel or other lumen can be intermittent or localized. Thus, for example, the vessel or other lumen can be stretched by about 4 to 50% (e.g., about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50%, percentages between the foregoing, etc.) along locations where the windings contact the vessel or lumen, but much less than 4% (none at all, 0-4%, etc.) at locations between the adjacent windings or elements of implant. In other embodiments, the vessel or other lumen can be stretched by more than about 50% (e.g., 50-60, 60-70, 70-80, 80-90, 90-100%, percentages between the foregoing, greater than about 100%, etc.), in the radial (e.g., circumferential) and/or axial (e.g., longitudinal) directions.

Accordingly, in some embodiments, the implant can result in selective “tenting” of the adjacent tissue of the vessel or lumen. One embodiment of such a tenting effect is illustrated in the perspective cross-sectional view of FIG. 4. In the depicted embodiment, distention of the vessel or lumen is provided according to length of stretching (in mm). In some embodiments, in order for the vessel or other lumen to accommodate the radial expansion from the implant (e.g., schematically depicted by the arrows), vessel or lumen tissue located between the adjacent windings or elements is pulled, stretched otherwise physically altered. According to some embodiments, bidirectional (e.g., radial and/or longitudinal) stretching of a vessel or other lumen resulting from a deployed implant can advantageously disrupt electrical condition and/or neural activity within, along or near the vessel or lumen. One embodiment of an implant configured to provide both radial and axial stretching to a targeted vessel or other lumen is illustrated in FIGS. 5A and 5B.

In any of the embodiments disclosed herein, the stretching of the lumen in the radial and/or axial directions can be intermittent and/or variable along the portion of the lumen affected by the implant. For example, the portions of the lumen that are contacted by the windings or elements of the implant can experience a greater degree of radial stretching that adjacent portions of the lumen (e.g., the areas between adjacent windings or elements), whereas the areas between adjacent windings or elements of the implant can experience a greater degree of axial stretching (e.g., as a result of the tenting effect caused by the implant). In some embodiments, the amount of stretching occurring in the radial direction is generally equal to, greater than or less than the amount of stretching occurring in the axial direction. For example, in some embodiments, after deployment of an implant within the target lumen, the lumen is stretched by about 20-30% in the axial direction and about 15-25% in the radial direction.

### Implant Designs

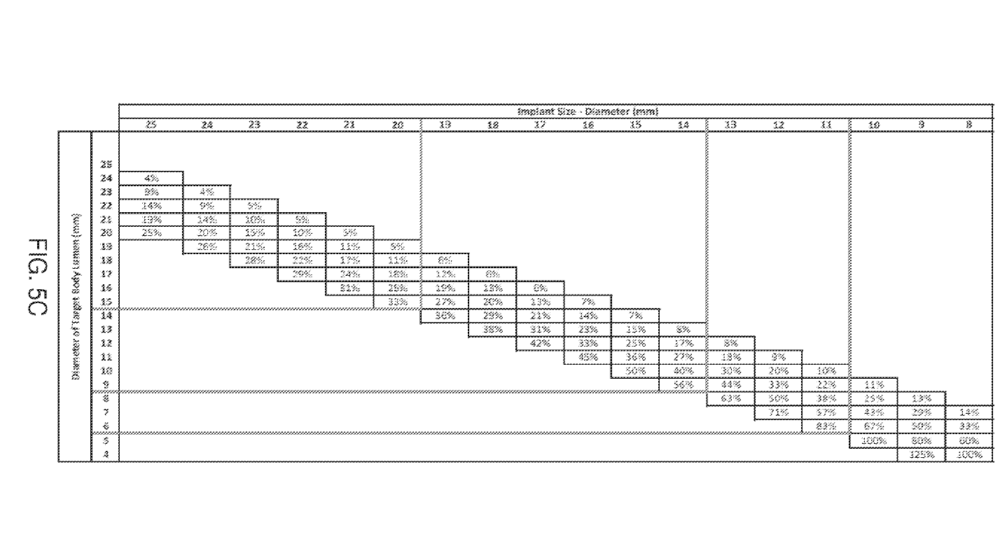

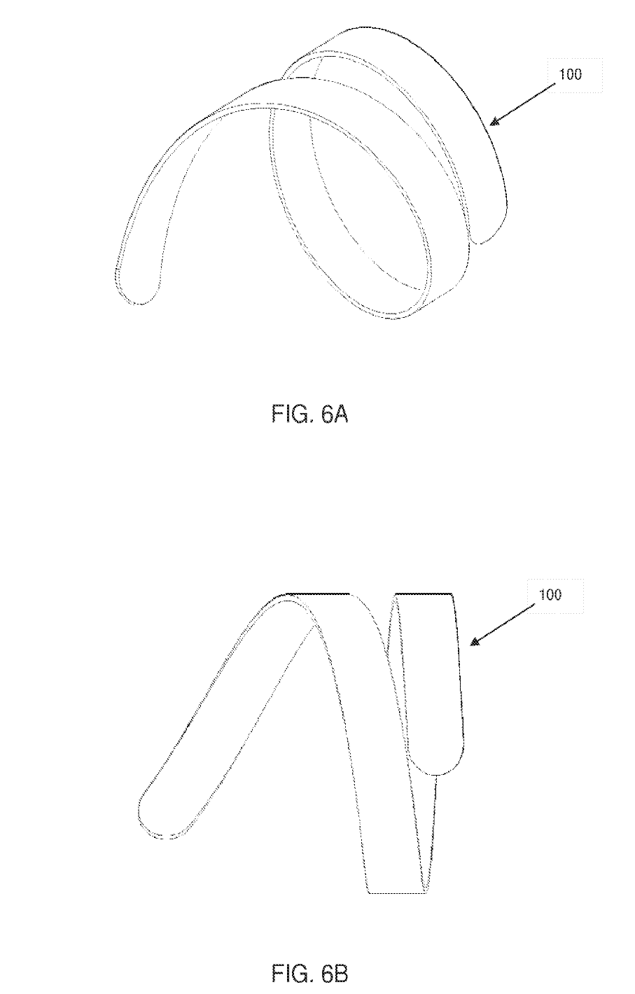

Various embodiments of an implant configured for placement within a vessel or other body lumen to cause the desired stress or stretching (e.g., in the radial and/or longitudinal directions) for disruption of electrical and/or neural propagation or activity along the vessel or other lumen are illustrated in FIGS. 6A to 20H. In any of the depicted embodiments, the implants can include a ribbon having a generally rectangular cross-sectional area (e.g., a cross section with angles that are between 85 degrees to 95 degrees, including a rectangle with 90 degree angles). In some embodiments, the ribbon comprises a smooth (e.g., flat) outer surface that is oriented and/or otherwise configured to contact and impart a force on adjacent vessel or lumen tissue when positioned and deployed therein. In some embodiments, such a smooth outer surface does not comprise any penetrating elements or features, so that once deployed within a target vessel or other body lumen, the implant exerts a pressure on adjacent vessel or other lumen tissue without penetrating such tissue. In some embodiments, the width of the ribbon is 0.5 to 8 mm (e.g., 0.5 mm, 1 mm, 1.5 mm, 2 mm, 2.5 mm, 3 mm, 3.5 mm, 4 mm, 4.5 mm, 5 mm, 5.5 mm, 6 mm, 6.5 mm, 7 mm, 7.5 mm, 8 mm, values between the foregoing, etc.). However, in other embodiments, the width of the ribbon is greater than 8 mm (e.g., 8.5 mm, 9 mm, 9.5 mm, 10 mm, 10-12 mm, 12-14 mm, 14-16 mm, 16-18 mm, 18-20 mm, greater than 20 mm, etc.), as desired or required. Forces delivered and/or pressures exerted by the deployed implant on the target tissue (e.g., a vessel wall) include, but are not limited to, 0.001 N/mm² to 3.5 N/mm² (e.g., between about 0.25 N/mm² and 1.5 N/mm²).

In any of the embodiments disclosed herein, the ribbon of the implant can include a rectangular cross-sectional shape with smooth outer surfaces. For example, the ribbon can include a width and a thickness. The outer surfaces of the ribbon can be smooth or generally smooth (e.g., free of any penetrating features or portions). In some embodiments, a ribbon can comprise generally 90-degree (e.g., generally sharp or abrupt) corners. In some embodiments, the use of such corners can help reduce the likelihood of migration of the implant relative to adjacent anatomical tissue after implantation. In some embodiments, the configuration of the implant reduces or prevents migration without the need for separate anchoring elements, such as anchoring legs, sutures, etc.

In some embodiments, however, the ribbon of the implant can include rounded corners or a different overall shape (e.g., rounded, circular or oval profile, along at least a portion of its cross-section), as desired or required. Regardless of the exact shape of the ribbon or other component or structure of an implant, the width of the ribbon can be larger than its thickness. In some embodiments, for example, the ratio of the width to the thickness is 1.5:1 to 30:1 (e.g., approximately 1.5:1, 2:1, 2.5:1, 3:1, 3.5:1, 4:1, 4.5:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 219:1, 30:1, values between the foregoing ranges, etc.). In other embodiments, the ratio of width to thickness of the ribbon or other structure can be less than 1.5:1 or greater than 30:1, as desired or required.

In some embodiments, the width of the ribbon is about 0.5 mm to 25 mm, e.g., 1 to 10 mm (e.g., about 1 mm, 1-1.1 mm, 1.1-1.2 mm, 1.2-1.3 mm, 1.3-1.4 mm, 1.4-1.5 mm, 1.5-1.6 mm, 1.6-1.7 mm, 1.7-1.8 mm, 1.8-1.9 mm, 1.9-2 mm, 2.1-2.2 mm, 2.2-2.3 mm, 2.3-2.4 mm, 2.4-2.5 mm, 2.5-2.6 mm, 2.6-2.7 mm, 2.7-2.8 mm, 2.8-2.9 mm, 2.9-3 mm, 3-3.1 mm, 3.1-3.2 mm, 3.2-3.3 mm, 3.3-3.4 mm, 3.4-3.5 mm, 3.5-3.6 mm, 3.6-3.7 mm, 3.7-3.8 mm, 3.8-3.9 mm, 3.9-4 mm, 4-4.1 mm, 4.1-4.2 mm, 4.2-4.3 mm, 4.3-4.4 mm, 4.4-4.5 mm, 4.5-4.6 mm, 4.6-4.7 mm, 4.7-4.8 mm, 4.8-4.9 mm, 4.9-5 mm, 5-5.5 mm, 5.5-6 mm, 6-6.5 mm, 6.5-7 mm, 7-7.5 mm, 7.5-8 mm, 8-8.5 mm, 8.5-9 mm, 9-9.5 mm, 9.5-10 mm, values between the foregoing ranges, etc.), e.g., 10-20 mm (e.g., 10-11 mm, 11-12 mm, 12-13 mm, 13-14 mm, 14-15 mm, 15-16 mm, 16-17 mm, 17-18 mm, 18-19 mm, 19-20 mm, 20-21 mm, 21-22 mm, 22-23 mm, 23-24 mm, 24-25 mm, values between the foregoing ranges, etc.) and/or the like. In other embodiments, the width of the ribbon is less that 0.5 mm or greater than 25 mm (e.g., 25-30 mm, 30-35 mm, 35-40 mm, 40-45 mm, 45-50 mm, greater than 50 mm, etc.). According to some embodiments, the thickness of the ribbon is 0.1 mm to 0.5 mm (e.g., 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5 mm, values between the foregoing, etc.). In other embodiments, however, the thickness of the ribbon is less than about 0.1 mm (e.g., 0.01, 0.02, 0.03, 0.04, 0.05, 0.06, 0.07, 0.08, 0.09, etc.) or greater than 0.5 mm (e.g., 0.5-0.6, 0.6-0.7, 0.7-0.8, 0.8-0.9, 0.9-1, 1-2 mm, more than 2 mm, etc.), as desired or required.

Further, in any of the implant embodiments disclosed herein, the overall length, diameter and/or other dimensions of the implants can vary, as desired or required. For example, in some embodiments, the diameter of the deployed (e.g., implanted) implant can vary between about 1 mm and about 50 mm (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50 mm, diameters between the foregoing values or ranges, etc.). In other embodiments, the diameter of the implant can be less than about 1 mm or greater than about 50 mm. In addition, for any of the implant embodiments disclosed or referenced herein, the implant can include any combination of diameter (e.g., or other cross-sectional dimension), ribbon width, ribbon thickness, ribbon width to thickness ratio, length and/or other dimensional property.

According to some embodiments, the aspect ratio of the implant can be selected to achieve a desired level of lateral stability (e.g., the implant's ability to maintain its longitudinal orientation within the lumen after implantation). The aspect ratio is the ratio of implant length to diameter. Thus, for a given diameter and overall shape, the longer the implant, the more stable it is expected to be within the subject's body lumen. Further, in some embodiments, an implant having a higher aspect ratio may be selected when the width of the ribbon is reduced in order to maintain a desired level of lateral stability post-implantation. For example: an aspect ratio of at least 1.5:1 may be used to attain a desired level of lateral stability for an implant having a ribbon width of about 1.5 mm, whereas a lower aspect ratio (e.g., of at least 1:1) can be used for an implant having a ribbon width of about 5 mm. Accordingly, implants can be provided in a variety of aspect ratios to permit a minimum level of lateral stability of the implant within the lumen to be maintained. In some embodiments, the aspect ratio can be modified by providing a tail or extension member to one end of the implant, as discussed in greater detail herein. In other embodiments, the aspect ratio of the implant can be increased by uniformly or generally uniformly lengthening the implant and/or by reducing the diameter of the implant. According to some embodiments, the implant can comprise an aspect ratio of between about 0.1:1 to about 10:1. For example, the implant can include an aspect ratio of 0.5:1 to 3:1 (e.g., 0.5:1, 0.6:1, 0.7:1, 0.8:1, 0.9:1, 1:1, 1.1:1, 1.2:1, 1.3:1, 1.4:1, 1.5:1, 1.6:1, 1.7:1, 1.8:1, 1.9:1, 2:1, 2.1:1, 2.2:1, 2.3:1, 2.4:1, 2.5:1, 2.6:1, 2.7:1, 2.8:1, 2.9:1, 3:1, 3.1:1, 3.2:1, 3.3:1, 3.4:1, 3.5:1, 3.6:1, 3.7:1, 3.8:1, 3.9:1, 4:1, 4.1:1, 4.2:1, 4.3:1, 4.4:1, 4.5:1, 4.6:1, 4.7:1, 4.8:1, 4.9:1, 5:1, ratios between the foregoing values, etc.). In other embodiments, the aspect ratio is less about 0.5:1 (e.g., between 0.1:1 and 0.5:1) or greater than about 5:1 (e.g., 5:1-6:1, 6:1-7:1, 7:1-8:1, 8:1-9:1, 9:1-10:1, greater than 10:1, etc.), as desired or required.

In some embodiments, once the implant has been released and implanted within a target vessel or body lumen (e.g., pulmonary vein, renal artery, etc.) or other anatomical location of a subject, the outer surface of the ribbon or other structure of the implant (e.g., along a width of the ribbon) can be generally parallel to the adjacent tissue of the subject (e.g., the interior wall of the vein, vessel or other lumen). In some embodiments, the implant is designed and otherwise configured so that the ribbon will be generally parallel to the adjacent anatomical tissue along an entire length or substantially an entire length of the implant, once the implanted has been deployed and implanted. Thus, the outer surface of the implant (e.g., the outer surface of the ribbon along its width) will apply a pressure to the adjacent tissue without penetrating the tissue.

According to some embodiments, an implant can include a single ribbon that comprises, at least in part, a generally helical or curved shape; however, in other embodiments, an implant can include more than one ribbon or structure. In some embodiments, the space separating adjacent windings or revolutions of a ribbon or other structure can vary and may also be a function of the overall length. In some embodiments, the ribbon includes at least one revolution or 360 degrees of a coil turn; however, in other embodiments the ribbon includes more or less than one revolution, as desired or required. The implant can include an “open” design, such that adjacent portions of the ribbon along the ring or ring portion do not contact one another. However, in some embodiments, the ribbon or other structure is configured to contact itself at least partially and/or intermittently along at least a portion of the implant. According to some embodiments, the pitch of the windings, revolutions or coils of a ribbon or other structure can vary. For example, the pitch or spacing of adjacent winding can be about 1-10 mm (e.g., about 1, 2, 3, 4, 5, 6, 7, 8, 9, 10 mm, values between the foregoing, etc.) pitch per turn. In other embodiments, however, the pitch is greater than 10 mm pitch per turn, such as, for example, 10-100 mm (e.g., 10-15 mm, 15-20 mm, 20-25 mm, 25-30 mm, 30-35 mm, 35-40 mm, 40-45 mm, 45-50 mm, 50-60 mm, 60-70 mm, 70-80 mm, 80-90 mm, 90-100 mm, values between the foregoing, etc.) pitch per turn. According to some embodiments, the ribbon can comprise one or more shape memory materials (e.g., Nitinol), coatings (e.g., medicaments, pharmaceuticals, etc.) and/or the like.

FIGS. 6A and 6B illustrate an intraluminal implant 100 configured to selectively exert a force on and/or stretch (e.g., radially and/or longitudinally) a vessel or other body lumen within which it is positioned. As shown, the implant 100 can include a ribbon that is shaped in a partially helical or curved configuration. The ribbon of the implant 100 depicted in FIGS. 6A and 6B comprises between one and two revolutions. However, in other embodiments, the number of revolutions can be less than one (e.g., ¼, ½, ¾, values between the foregoing, less than ¼, more than ¾ and/or the like) or greater than two (e.g., 2½, 3, 4, values between the foregoing, more than 4, etc.), as desired or required. In addition, as illustrated by the embodiment of FIGS. 6A and 6B, the ribbon can be generally asymmetrical along the length of the implant (e.g., in the manner in which it is wound or shaped around a central axis). For example, the depicted implant comprises a tail or extension portion along one end. Thus, the pitch of the ribbon can be selectively varied along the length of the implant. This can help increase the stability of the implant, such that the likelihood of undesirable twisting or turning within the implant site (e.g., vessel, lumen, etc.) is reduced or eliminated.

In some embodiments, a tail portion or extension along one end of the implant can help ensure that a larger longitudinal extent of the targeted vessel or body lumen is covered. For example, the tail can extend deeper into a pulmonary vein, renal artery and/or any other vessel or lumen to help ensure that the desired electrical block and/or denervation is achieved. In some embodiment, the opposite end of the ribbon (e.g., the end having a reduced pitch or spacing between adjacent windings) can be configured to more securely affix the implant at or within a targeted site (e.g., at or near an ostium or other vessel opening, within a vessel, airway or other body lumen, etc.).

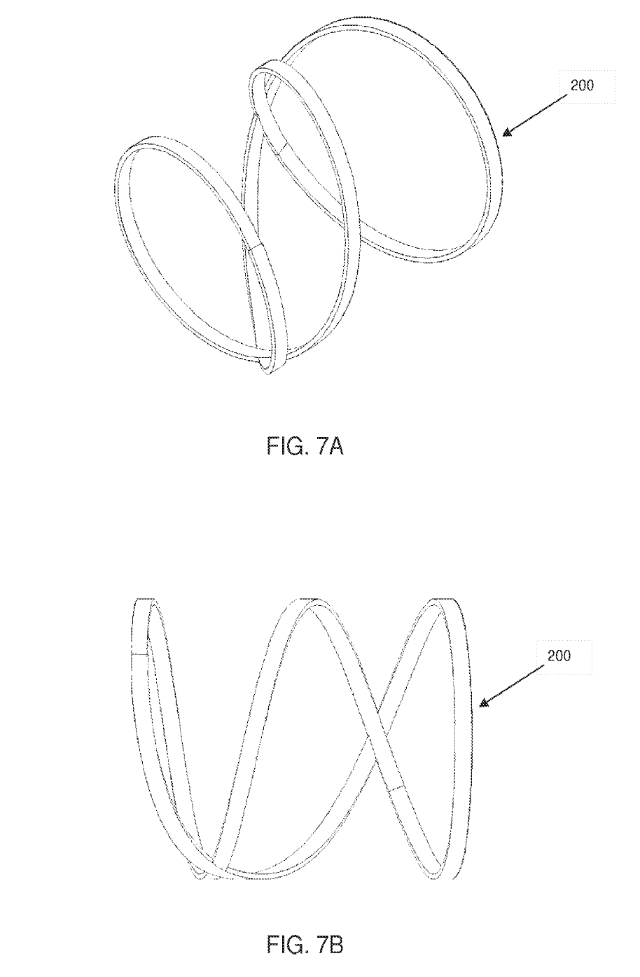

FIGS. 7A and 7B illustrate another embodiment of an implant 200 comprising a ribbon configured for placement within a target vessel or body lumen. As shown, the implant 200 includes a continuous ribbon that is configured to not include any free ends. In some embodiments, the implant comprises a single, continuous ribbon that is shaped into the desired configuration without any cuts, welds and/or other connections. In other embodiments, however, the implant 200 can include two or more (e.g., 3, 4, more than 4, etc.) separate ribbon portions that are selectively attached to one another using one or more connection devices or methods (e.g., fasteners, welds, adhesives, etc.).

With continued reference to the implant 200 of FIGS. 7A and 7B, the ribbon can include a double helix or similar design along at least a portion of the implant (e.g., along the middle or central portion of the implant). In some embodiments, such a design can result in a very stable shape, such that the implant will not undesirably turn or twist during or after delivery within the target vessel or other body lumen.

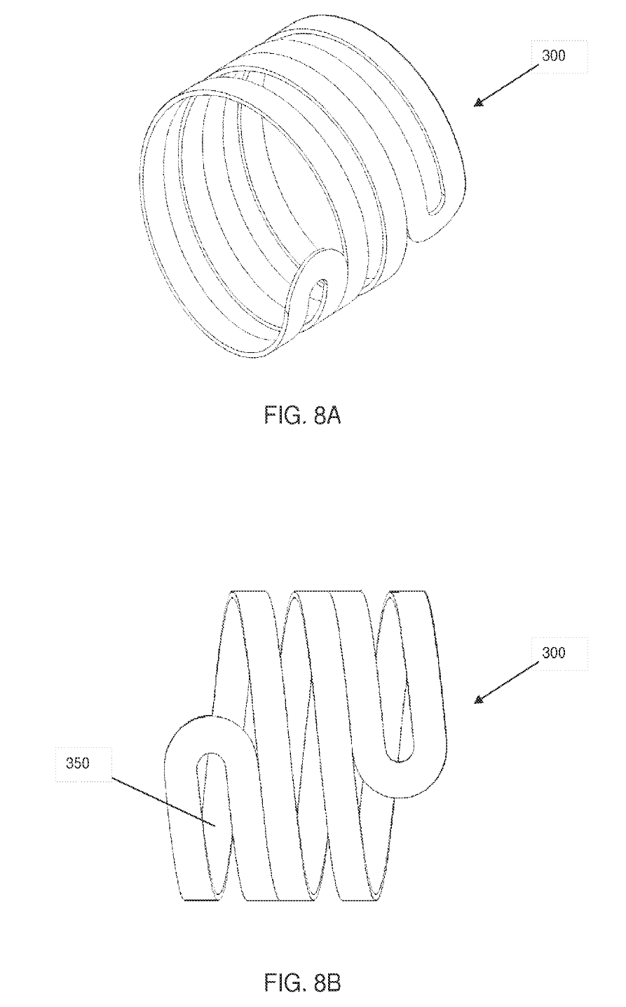

In some embodiments, as illustrated in FIGS. 8A and 8B, the ribbon of the implant 300 can include a central recess, slot or other opening that extends partially or completely through the length of the implant. As shown, the central recess or opening can extend continuously from one end of the implant to the other. However, in other embodiments, the recess or opening extends only intermittently and/or only partially along the ribbon, as desired or required. According to some embodiments, the recess or other opening can help increase the stability, strength and/or other physical properties of the resulting implant 300 (e.g., increase the implant's resistance to undesirably overturning, twisting or otherwise moving away from a desired axis or orientation during or after implantation). In some embodiments, an opening (e.g., slot or other recess) can advantageously reduce the total material needed to manufacture the implant and/or reduce the overall weight or mass of the implant. In some embodiments, the ribbon can be configured with one or more openings so that the force, pressure, degree of stretching, etc. caused by the resulting the implant can be within a target range or at or near a target value. Such openings (e.g., slots, slits, windows, etc.) can be originally formed with the ribbon or can be cut or otherwise made after the ribbon has been manufactured.

As illustrated in FIGS. 8A and 8B, the implant 300 can be configured so that adjacent windings of the ribbon are either touching or nearly touching one another. For example, in some embodiments, adjacent windings of the ribbon can be separated by 0 to 100 mm (e.g., 0-0.1, 0.1-0.25, 0.25-0.5, 0.5-1, 1-2, 2-3, 3-4, 4-5, 5-6, 6-7, 7-8, 8-9, 9-10, 10-15, 15-20, 20-25, 25-30, 30-35, 35-40, 40-45, 45-50, 50-60, 60-70, 70-80, 80-90, 90-100 mm, values between the foregoing ranges, etc.). Further, as shown, the ribbon can be configured so that adjacent windings have a generally constant or consistent pitch (e.g., angle relative to the longitudinal axis of the implant 300 or the vessel or body lumen within which the implant is positioned). In some embodiments, the ends of the ribbon comprise a generally rounded or curved shape, as illustrated in FIGS. 8A and 8B. However, in other embodiments, the ends can include a non-rounded or other shape or configuration (e.g., rectangular, other polygonal or sharp cornered design).

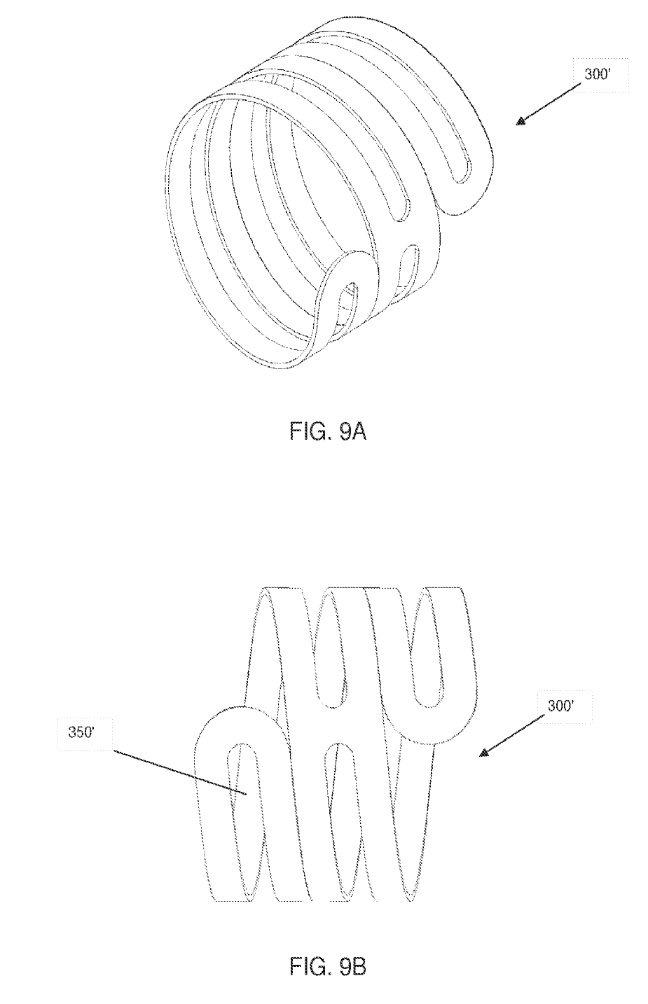

FIGS. 9A and 9B illustrate a different embodiment of an implant 300′ having a ribbon with an opening (e.g., one or more slots, pores, recesses, slits, etc.). However, unlike the implant of FIGS. 8A and 8B, the depicted embodiment comprises a non-continuous or interrupted slot or central opening. Alternatively, such a ribbon can be thought of having a generally continuous slot or recess with one or more struts or ribs that extent across, and thus, divide the slot or recess. In some embodiments, such a configuration can enhance the strength, stability and/or other physical properties of the implant 300′. Such slotted or “open” configurations of a ribbon can be incorporated into any of the embodiments disclosed herein or in any of the embodiments disclosed in the cross-referenced applications, including, without limitation, the various implants of U.S. patent application Ser. No. 13/830,040, filed Mar. 14, 2013. The openings of the ribbon can be continuous (e.g., as shown in FIGS. 8A and 8B) or discontinuous (e.g., having one or more struts, ribs and/or the like, as shown in FIGS. 9A and 9B).

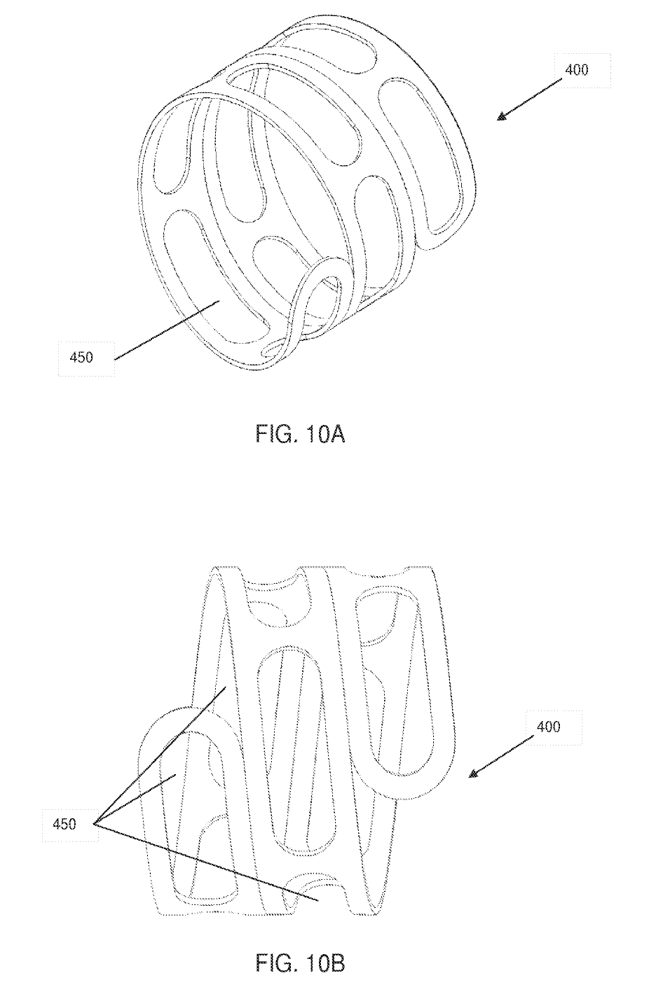

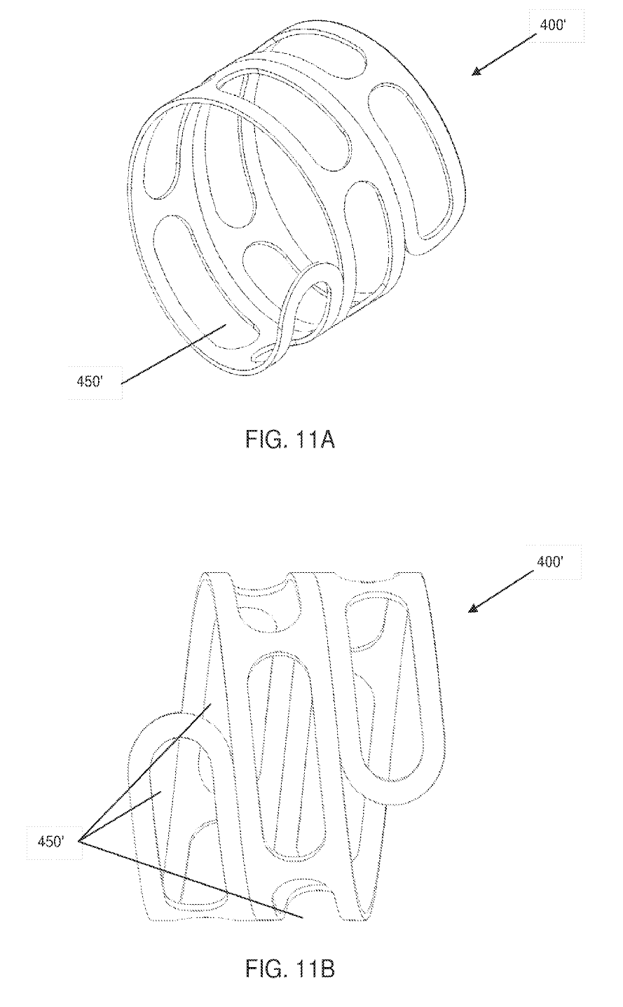

Additional embodiments of open-ribbon (e.g., slotted) implants are illustrated in FIGS. 10A, 10B, 11A and 11B. As shown, the ribbon can include one or more openings or windows 450, 450′ along the length of the implant 400, 400′. Such openings or windows can be originally included in the ribbon that is used to make the implant. Alternatively, the openings or windows can be created after the ribbon has been manufactured (e.g., by cutting out material form a solid piece of ribbon, by attaching one or more struts or ribs to an open ribbon design, etc.).

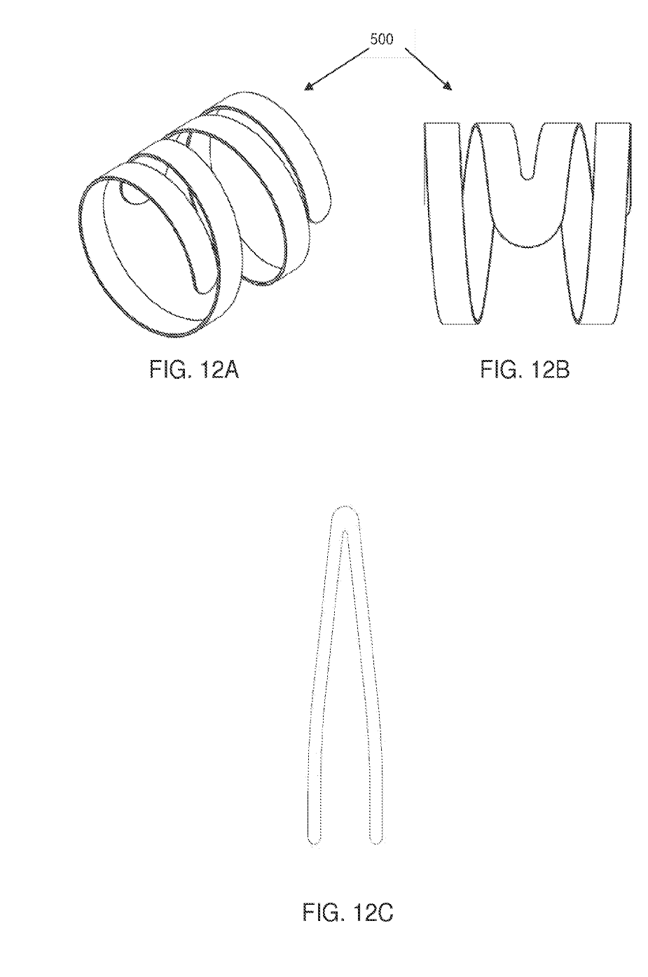

FIGS. 12A and 12B illustrate yet another embodiment of an implant 500 configured for placement within a vessel or other body lumen of a subject. In some embodiments, the implant 500 is manufactured by selectively shaping a frame having an “A” or “V” shape, as illustrated in FIG. 12C. For example, as with other embodiments disclosed and/or referenced herein, the frame can be formed and heat set around a mandrel and/or any other manufacturing method. The resulting implant can include favorable physical properties (e.g., strength, stability, etc.). In some embodiments, the resulting ribbon does not include a consistent and/or continuous pitch (or slope relative to the longitudinal axis of the implant). As shown, the implant 500 can include a central point of inflection, about which the ribbon is generally symmetrical. In addition, as shown, such an implant 500 can include free ends of A or V-shaped ribbon along each side of the formed implant.

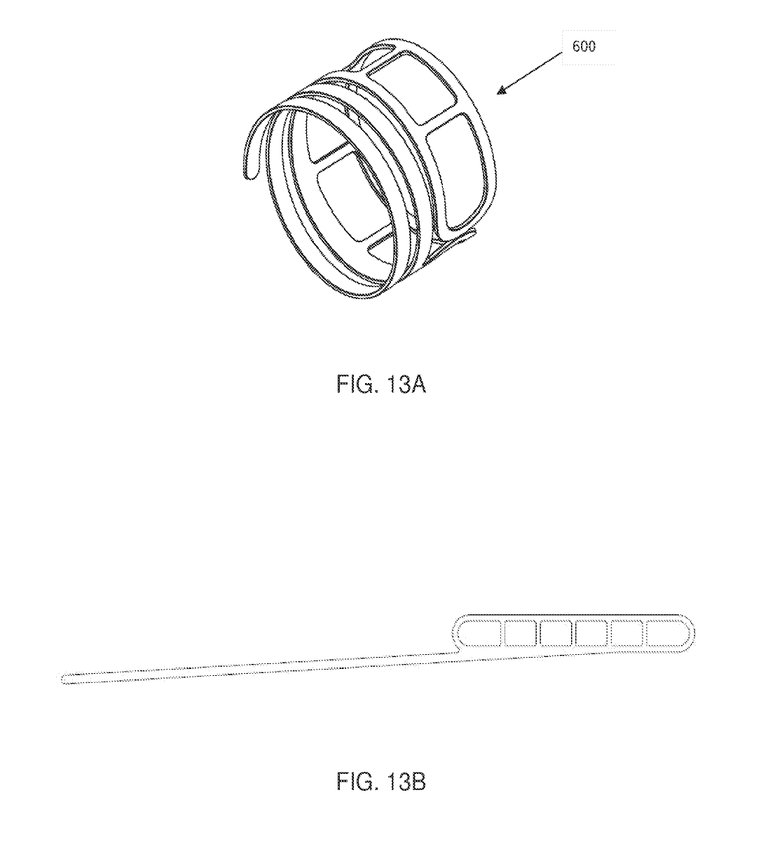

FIG. 13A illustrates a perspective view of yet another embodiment of an implant 600 configured for placement within a target vessel or other body lumen of a subject and configured to exert a force or stress along the vessel or lumen. As shown, the implant 600 comprises a ribbon having an open structure (e.g., a recess or central opening). In some embodiments, as depicted in FIG. 13A, the section of the ribbon having the recess, slot or other opening can extend only along one end (or along one or more portions) of the implant. As discussed with reference to other implants herein, the open (e.g., slotted) portion of the ribbon can be continuous or intermittent (e.g., having two or more windows, open areas, etc.), as desired or required.

With continued reference to FIG. 13A, the implant 600 can include a tail or other extension member that flares or protrudes along one end of the ribbon. In the depicted embodiment, the tail comprises a significantly thinner shape, but has an angle relative to the longitudinal axis of the implant that generally matches the angle of the open-ribbon portion. However, in other configurations, the thickness, angle, pitch and/or other details of the tail or other extension member, the open (e.g., slotted) portion of the ribbon and/or other property of the implant can vary, as desired or required.

According to some embodiments, as with other designs described herein, the implant 600 of FIG. 13A can be manufactured by selectively shaping a frame (e.g., heat setting a frame around a mandrel). For example, in one embodiment, the frame of FIG. 13B can be selectively shaped to form the implant illustrated in FIG. 13A.

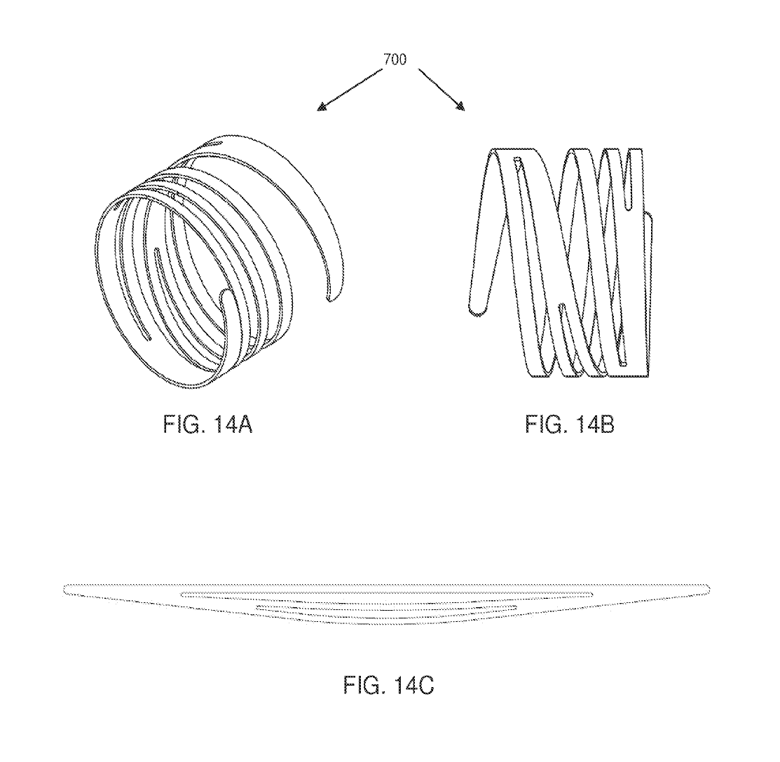

FIGS. 14A and 14B illustrate different views of another intraluminal (e.g., intravascular) implant 700 that is configured to exert a pressure along adjacent tissue of a subject upon implantation. As shown, the implant 700 can include a ribbon that is asymmetrically-shaped along a longitudinal axis of the implant. As with other embodiments disclosed or referenced herein, the ribbon can include one or more openings (e.g., slots) along its length. The extent (e.g., length), width, other dimensions, shape (e.g., oval, circular, irregular, rectangular with curved edges, etc.), orientation and/or details of such an opening can vary along the length of the ribbon. Such variability applies to any other implant design disclosed and/or referenced herein. In one embodiment, the frame of FIG. 14C can be selectively shaped to form the implant illustrated in FIGS. 14A and 14B. In some embodiments, the openings (e.g., slots) enhance the stability of the implant during and after implantation (e.g., reduce the likelihood of undesirable torqueing or twisting of the implant), help reduce the likelihood of migration of the implant after deployment within a target body lumen, facilitate tissue ingrowth (e.g., endothelialization) around the implant (e.g., to promote long-term fixation within the subject), provide for enhanced pressure distribution (e.g., of forces exerted on adjacent tissue) along the length of the implant and/or the like.

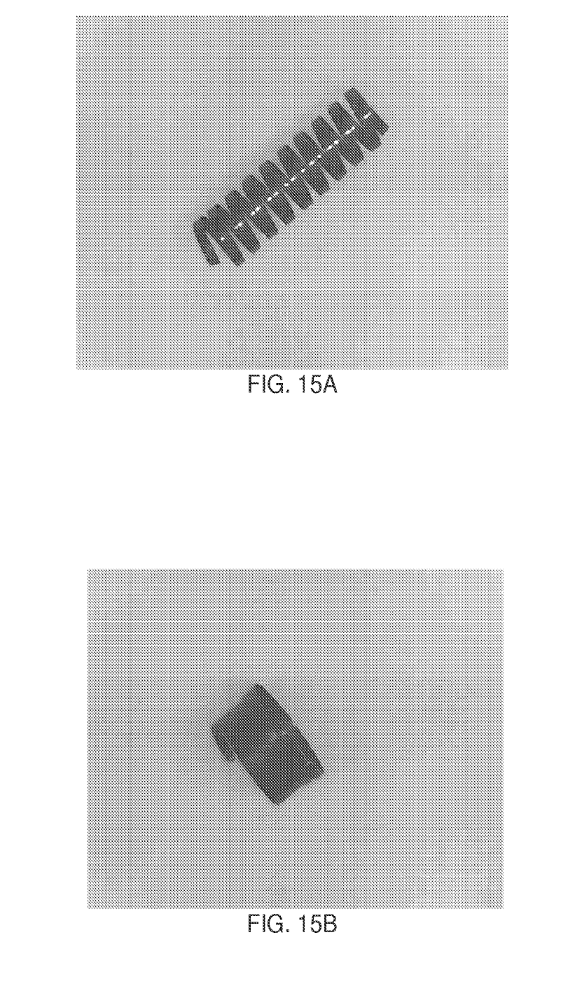

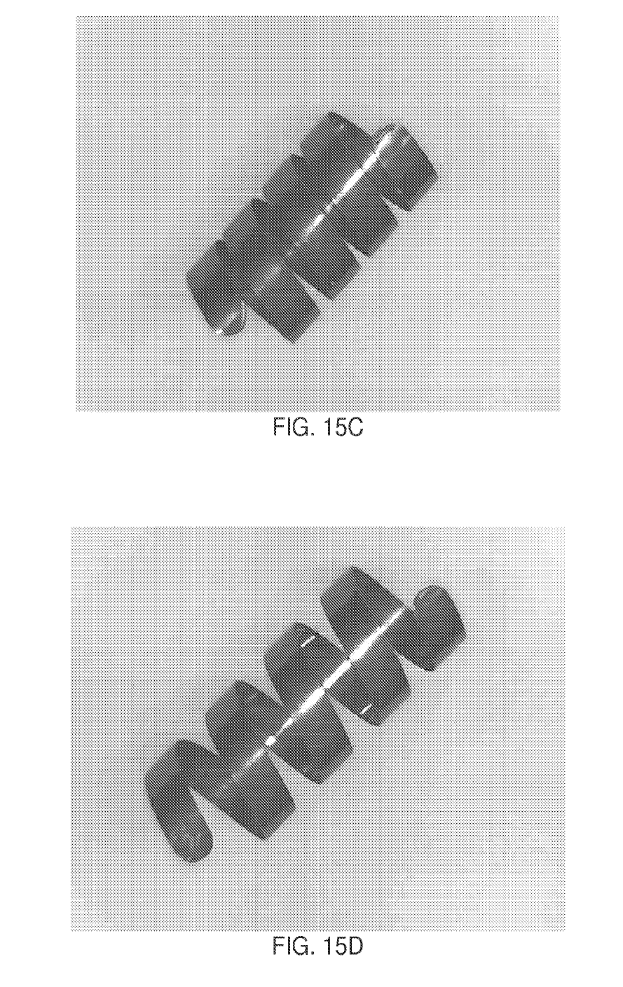

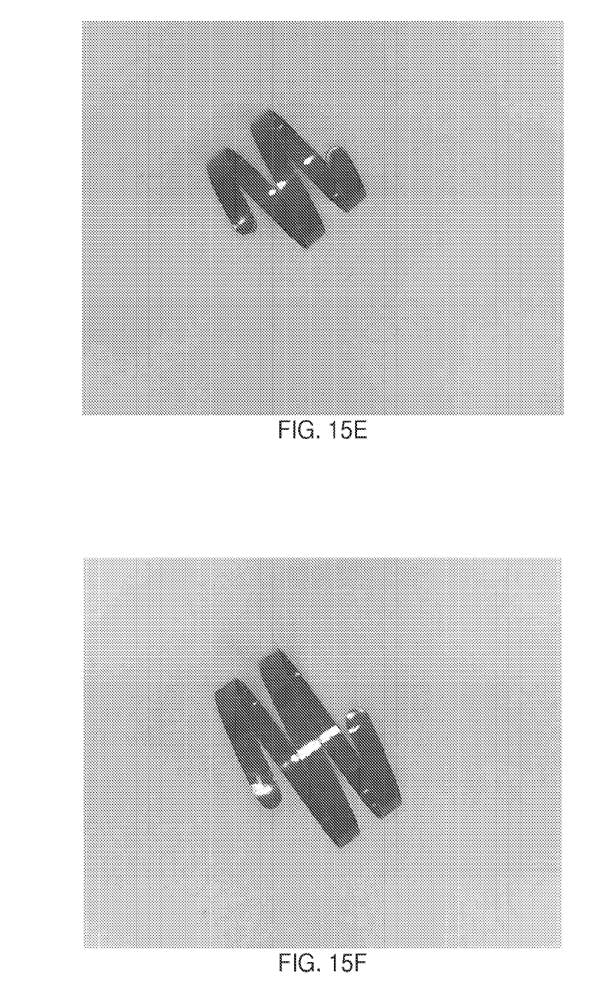

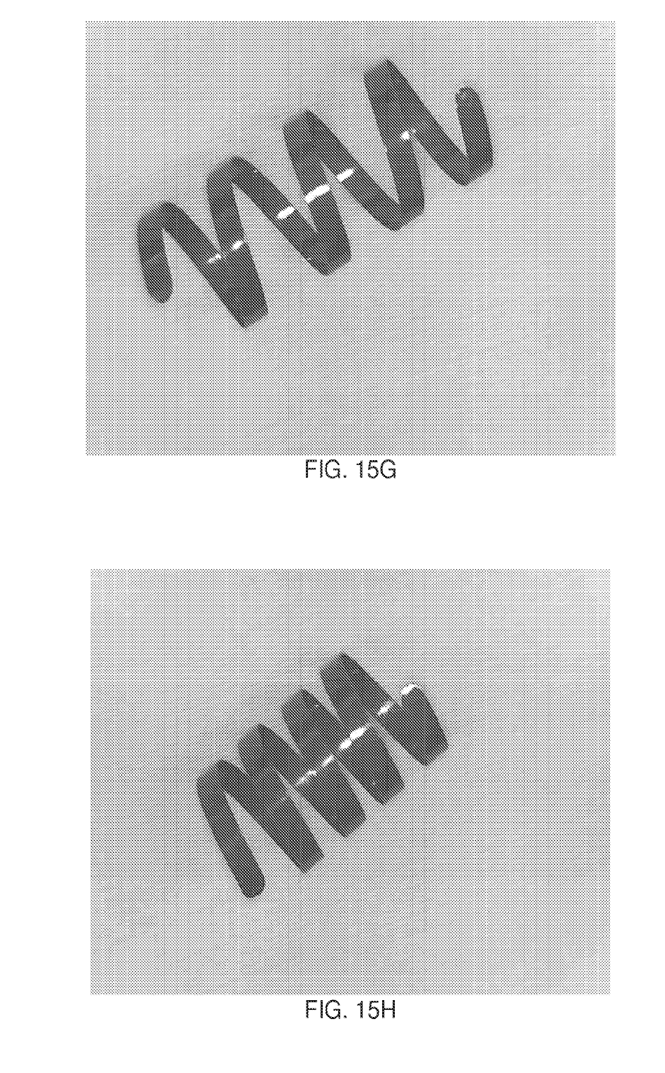

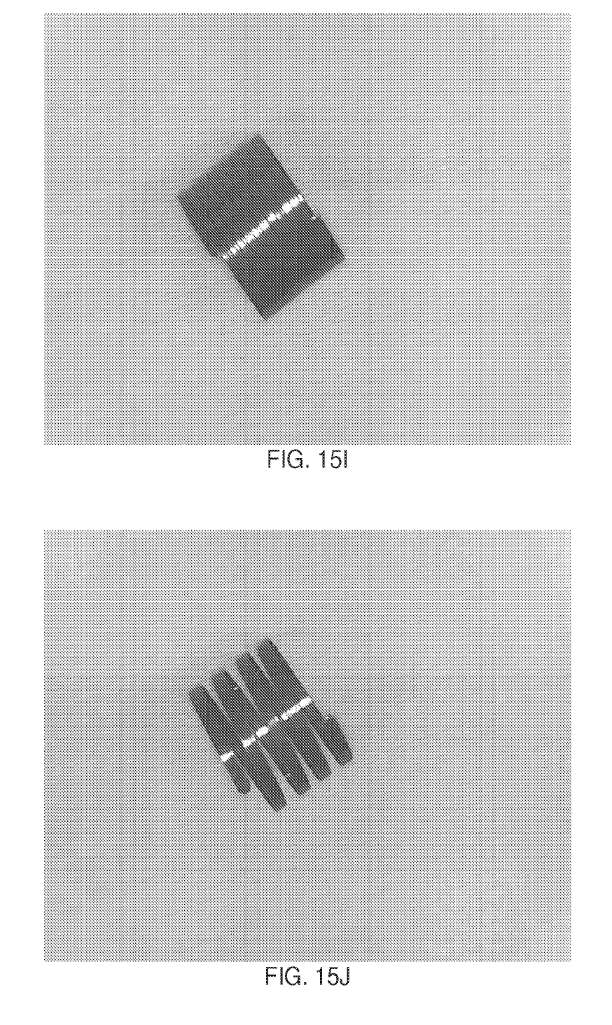

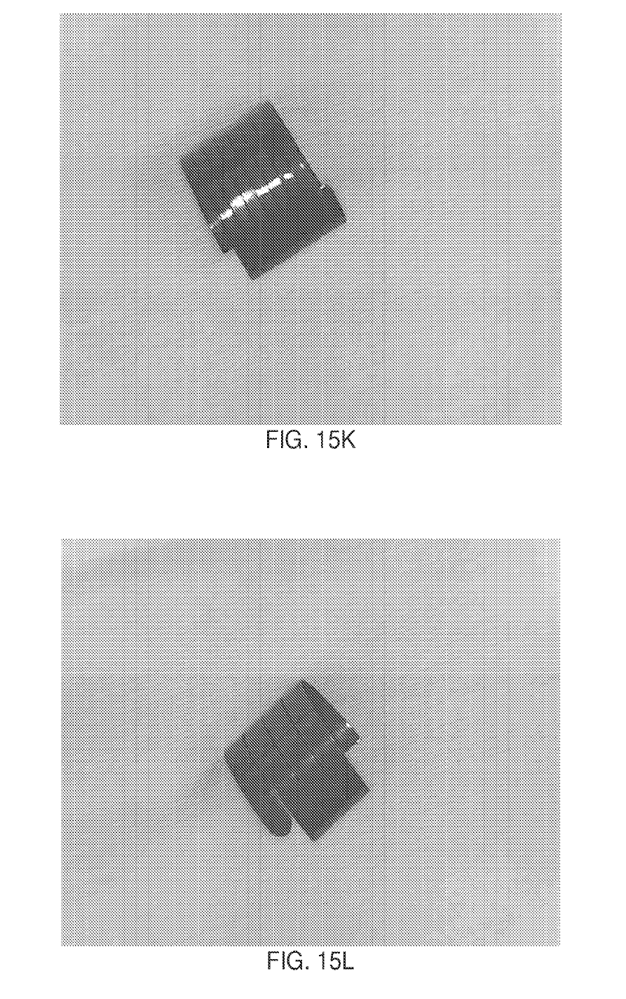

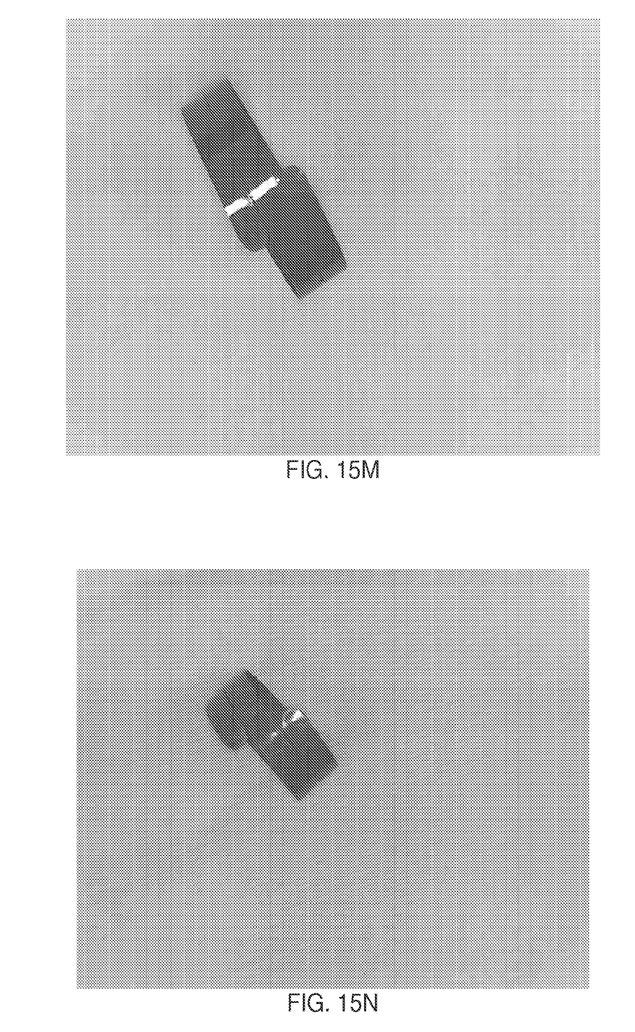

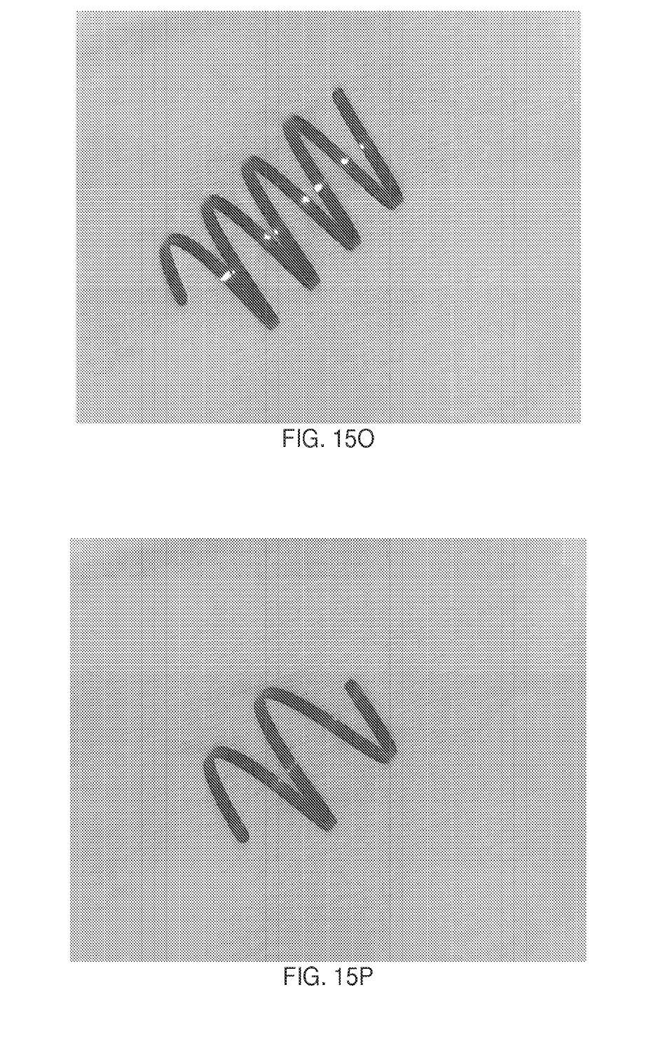

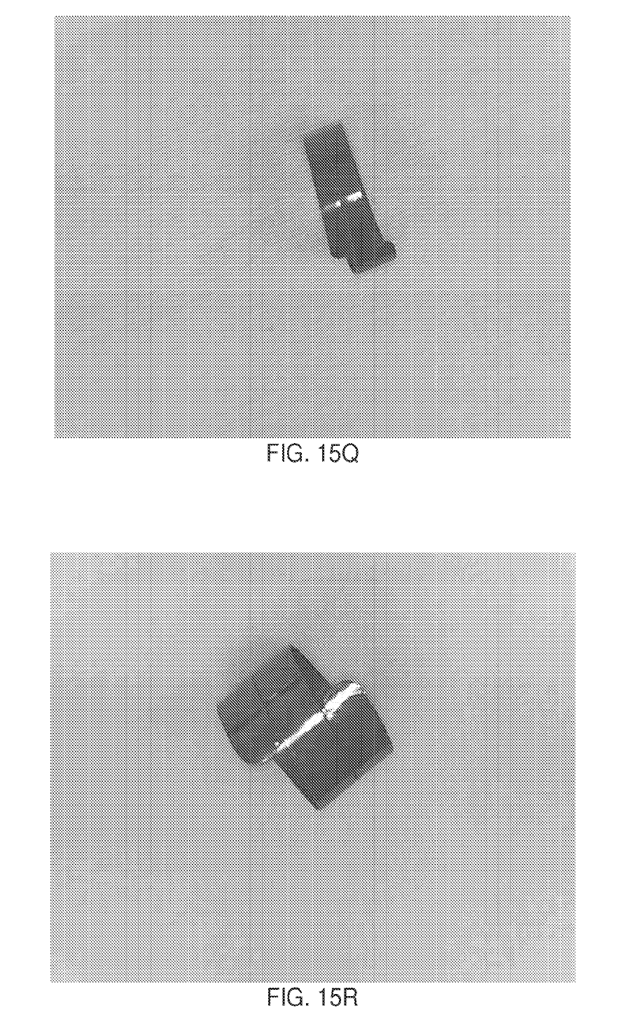

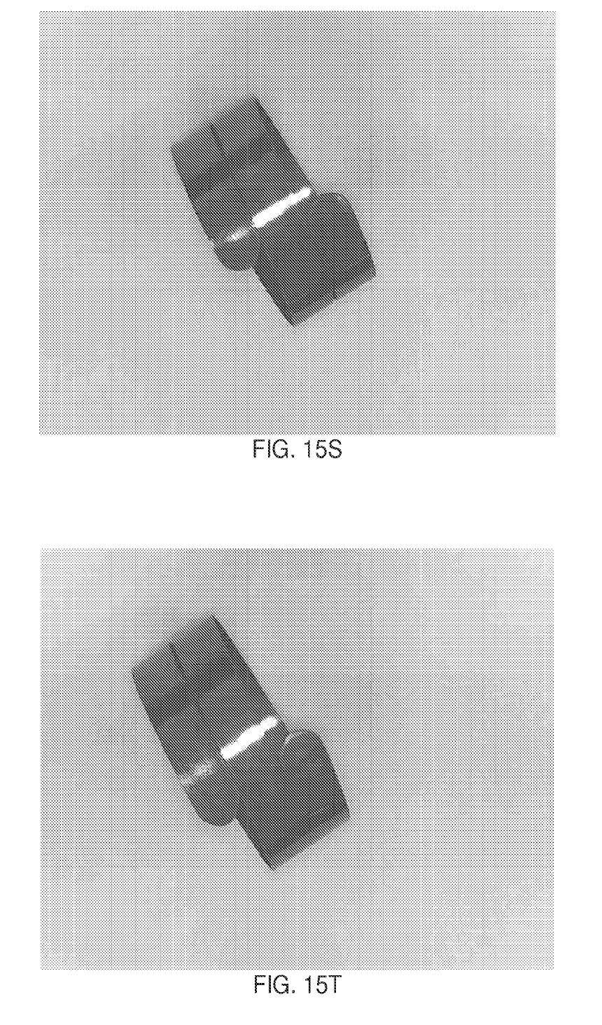

FIGS. 15A-15T illustrate a variety of other implant embodiments that include a generally consistent pitch (or angle of windings relative to the longitudinal axis) along their respective length. As shown, the implants can include no or virtual no space between adjacent windings or portions of a ribbon. In such embodiments, adjacent windings of the ribbon either touch or almost touch one another (e.g., are separated by a particular distance, spacing or clearance, such as, 0 to 2 mm, e.g., 0-0.1, 0.1-0.25, 0.25-0.5, 0.5-1, 1-2, 2-3, 3-4, 4-5, 5-6, 6-7, 7-8, 8-9, 9-10 mm, values between the foregoing ranges, etc.). See, for example, FIGS. 15B, 15I, 15K-15N and 15Q-15T. Alternatively, however, adjacent windings of the ribbon can be spaced apart so as to create larger openings between the windings (e.g., greater than 2 mm, e.g., 0.2-0.25, 0.25-0.5, 0.5-1, 1-2, 2-3, 3-4, 4-5, 5-6, 6-7, 7-8, 8-9, 9-10 mm, values between the foregoing ranges, etc.). See, for example, FIGS. 15A, 15C-15H, 15J, 15O and 15P. In addition, for any of the disclosed embodiments, the pitch or spacing can vary, as desired or required, to allow the implant to include a tail, an asymmetrical design and/or the like.

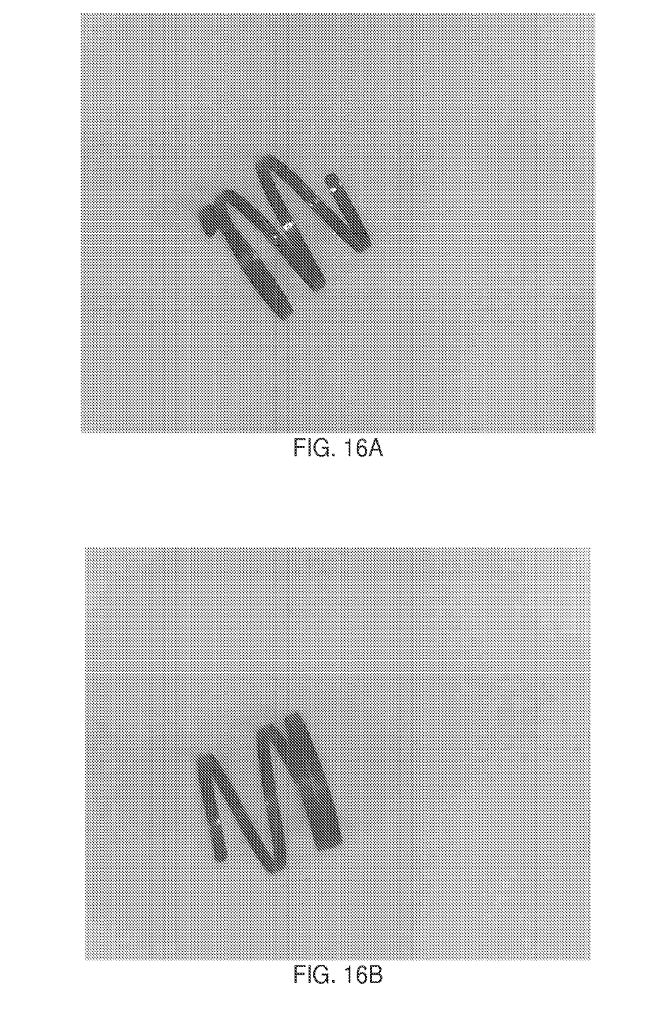

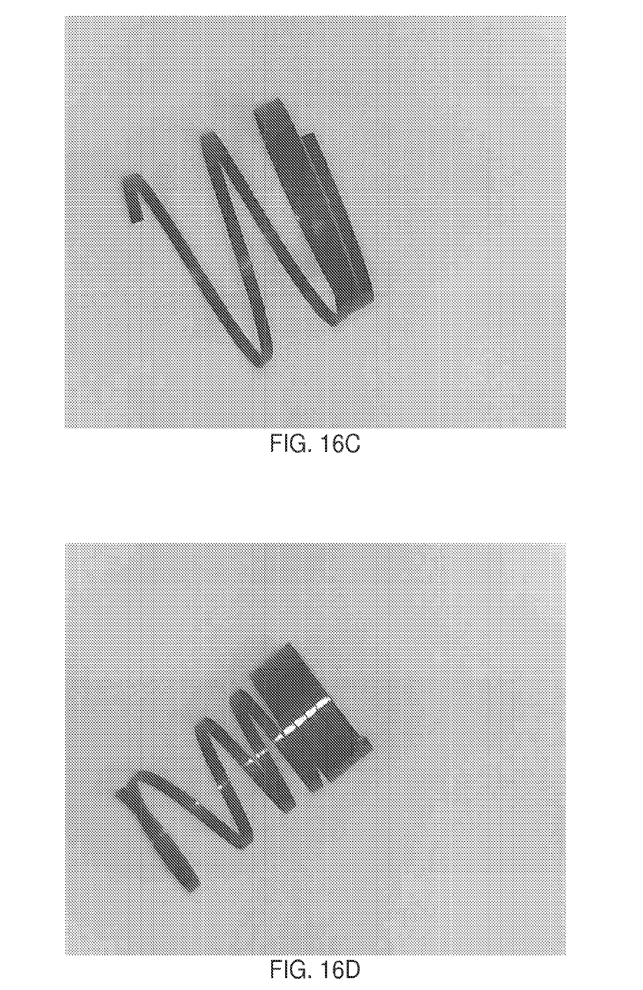

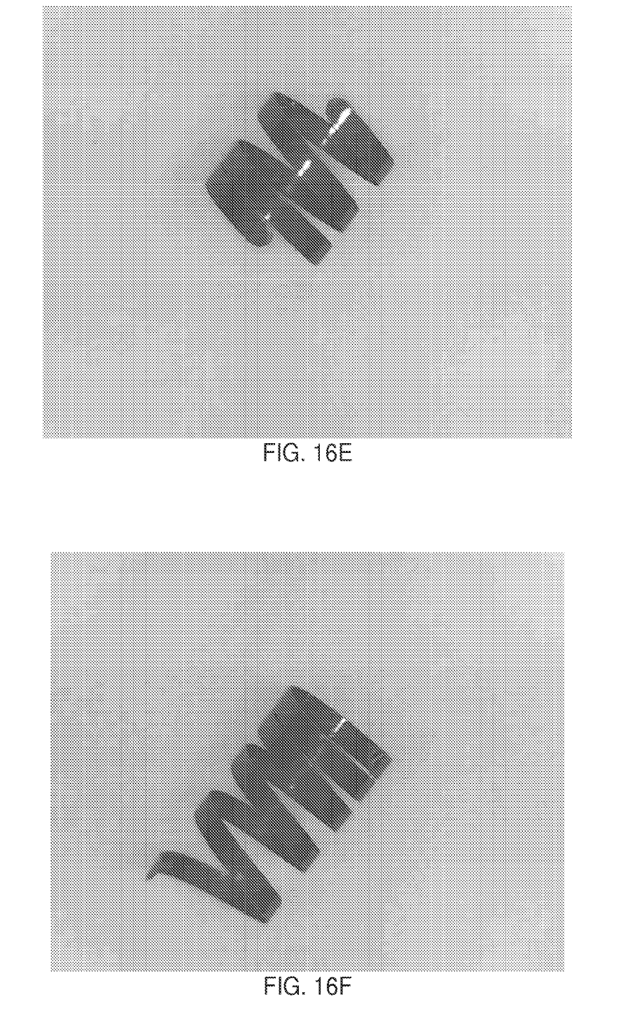

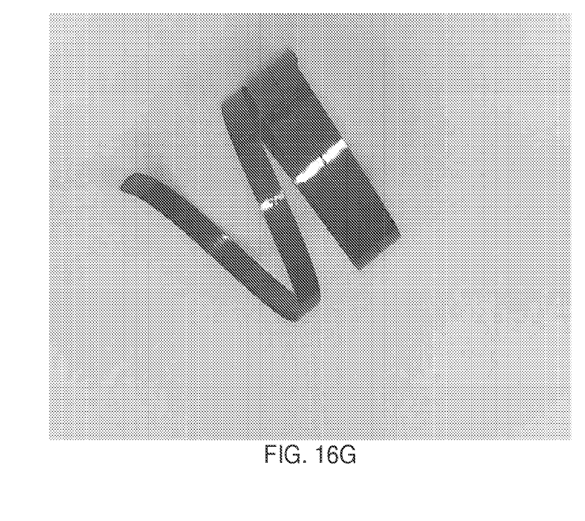

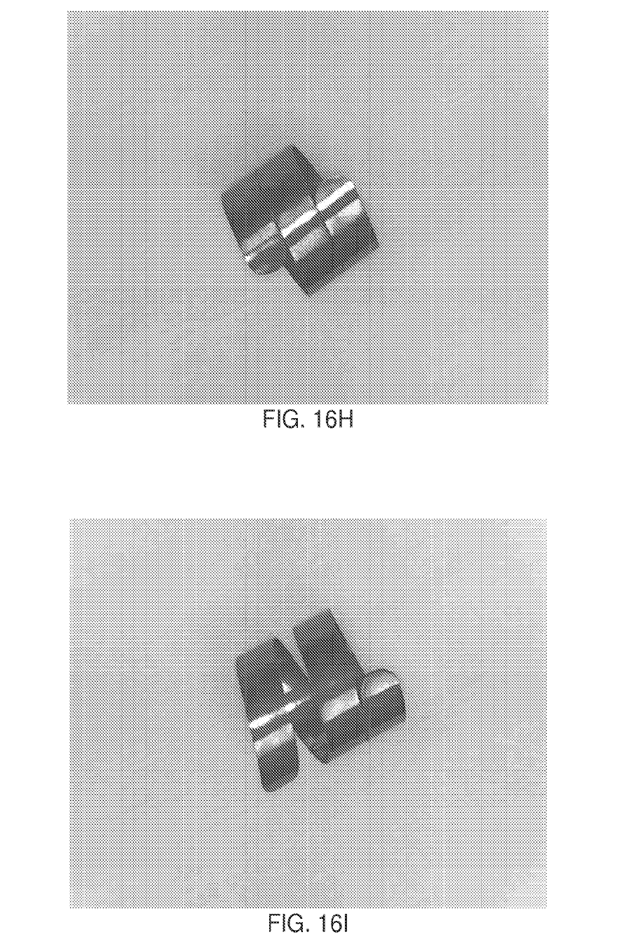

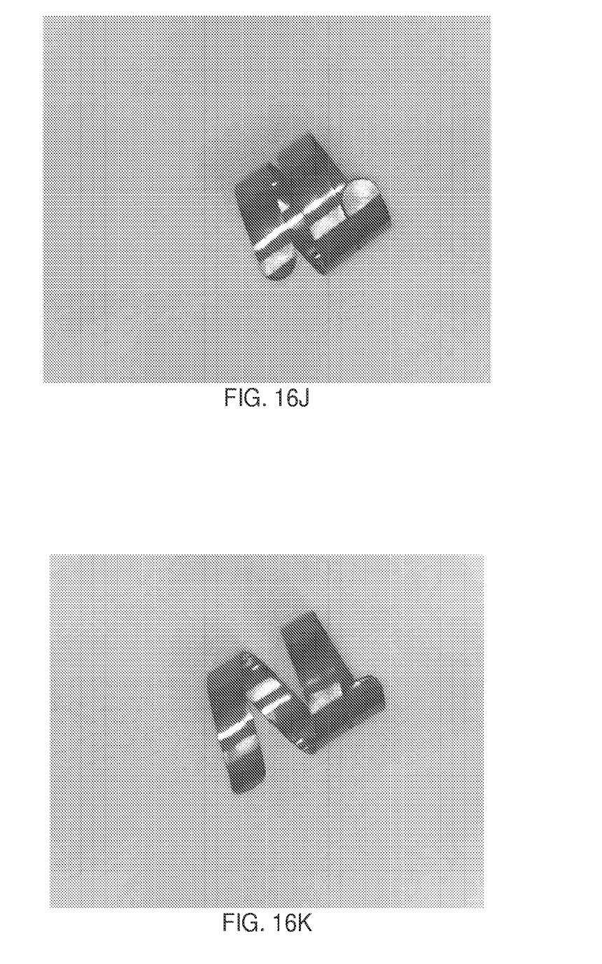

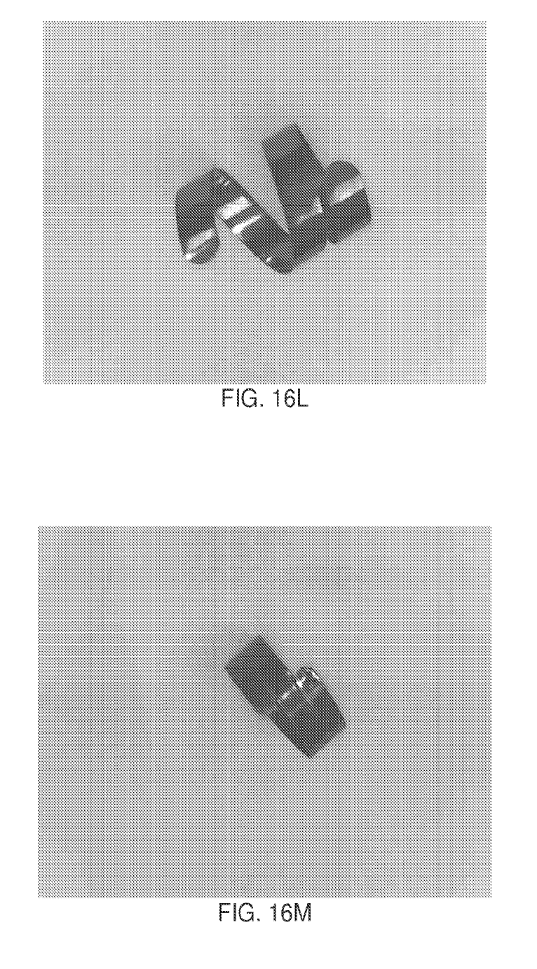

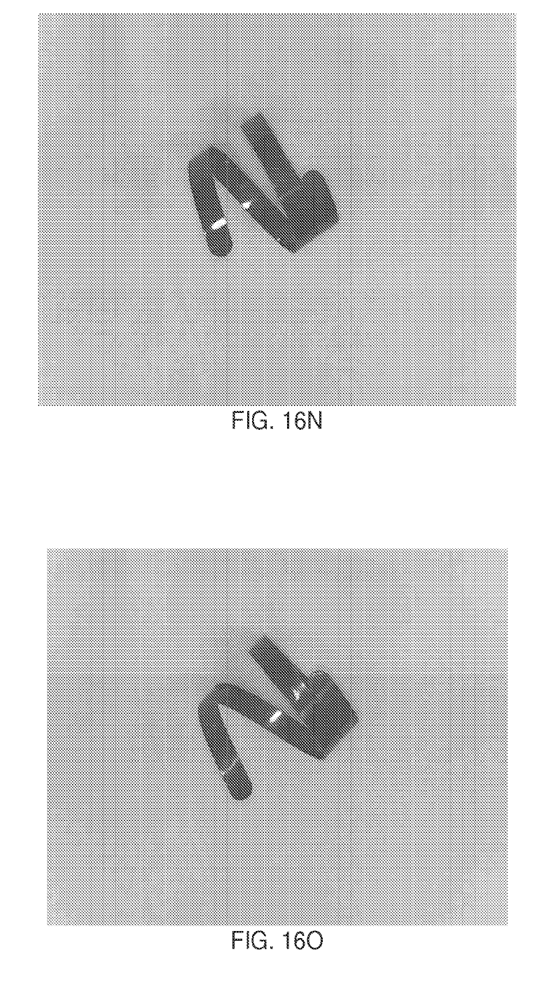

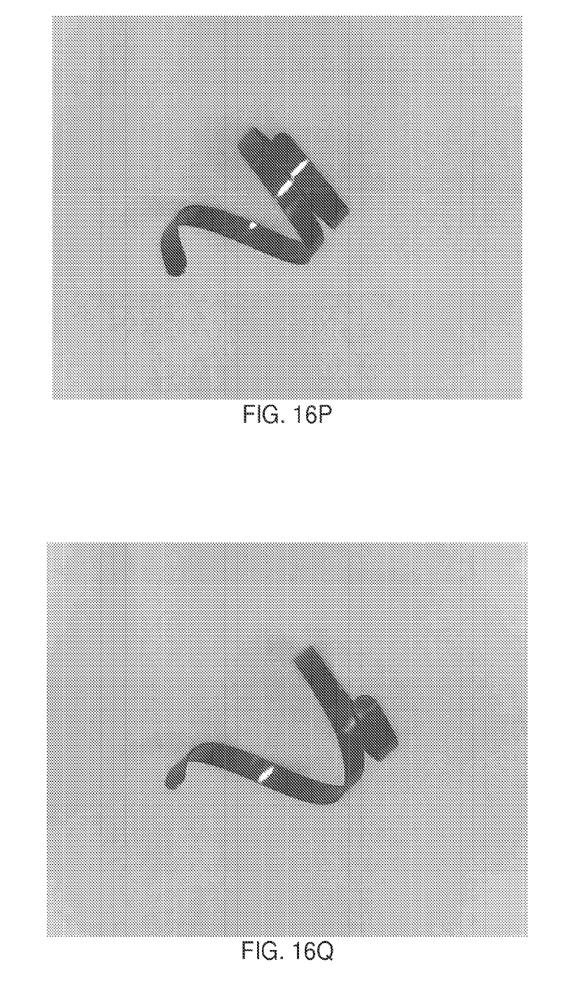

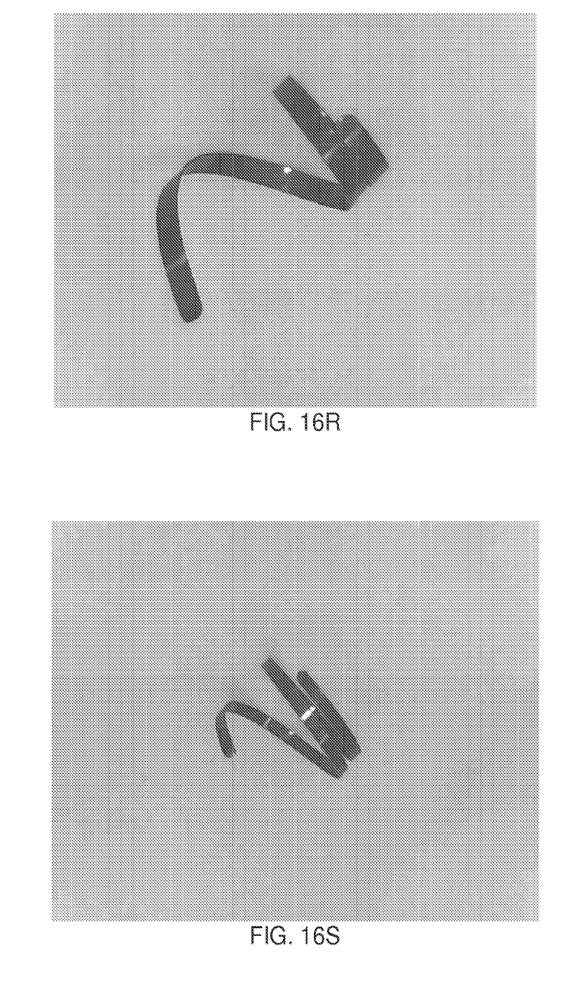

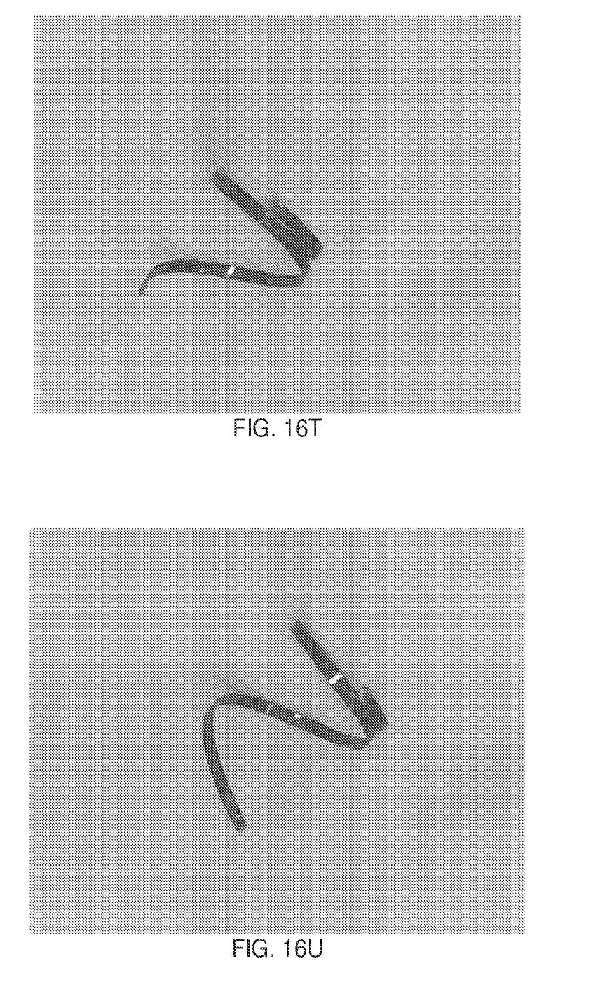

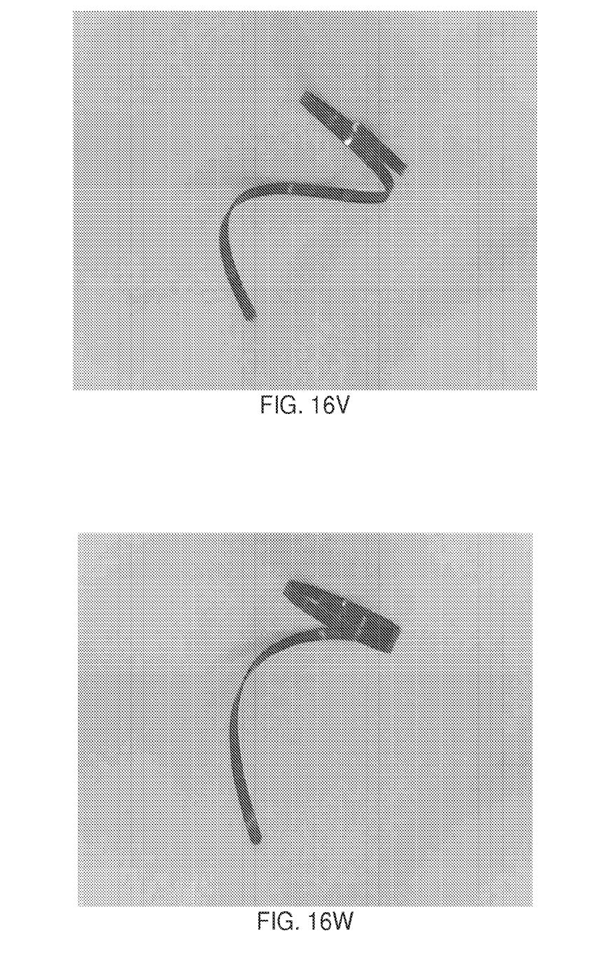

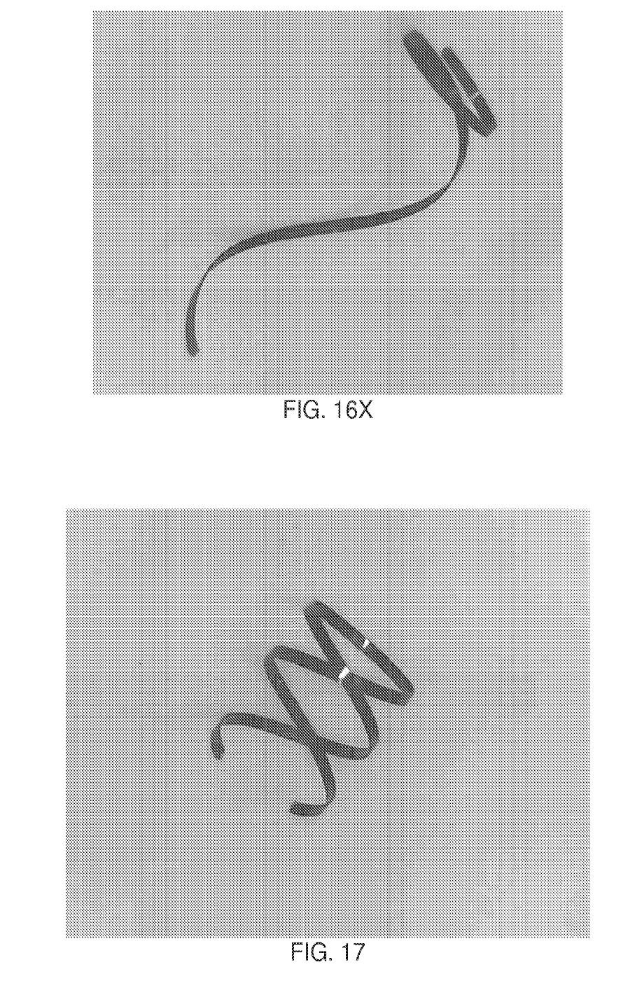

Examples of asymmetrical implant designs or implants comprising a tail or similar extension or feature are illustrated in FIGS. 16A-16X. As shown, and as discussed with other embodiments herein, the ribbon can be formed to create a desired spacing between adjacent windings. As noted herein, the aspect ratio (ratio of length to diameter) of the implant can vary (e.g., in order to achieve a particular lateral stability after implantation into a target lumen). In some embodiments, the aspect ratio is between 0.1:1 and 10:1 (e.g., 0.5:1 to 3:1). For example, the aspect ratios of the implants illustrated in FIGS. 16N, 16O, 16P, 16Q, 16R, 16S, 16T, 16U, 16V, 16W and 16X are 1:1, 1.25:1, 1.5:1, 1.75:1, 2:1, 8:1, 1.25:1, 1.5:1, 1.75:1, 2:1 and 3:1, respectively.

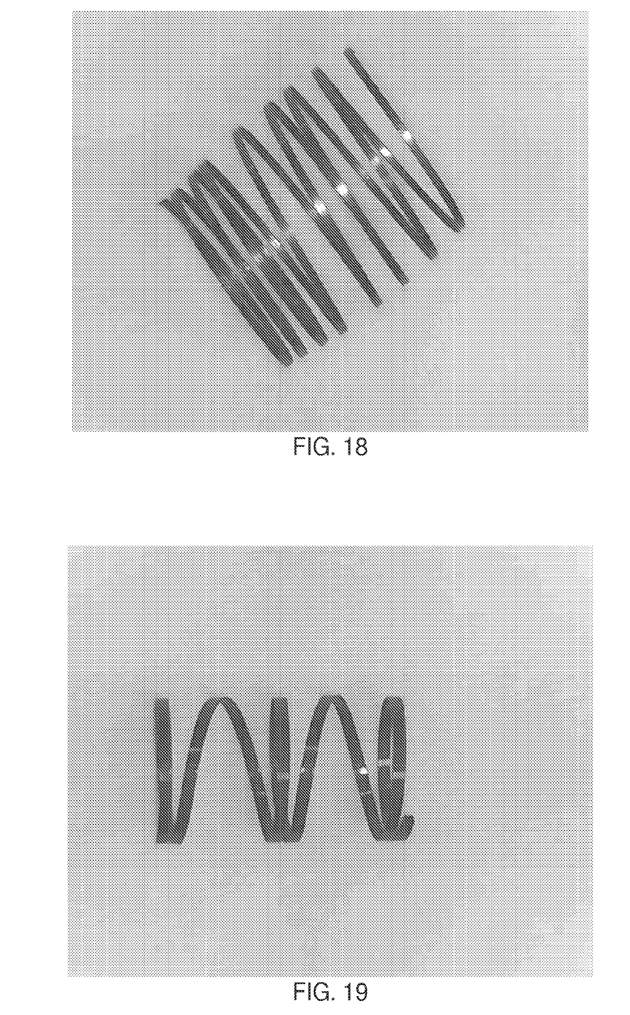

Another embodiment of an intraluminal implant is illustrated in FIG. 17. The depicted implant is similar to the implant of FIGS. 7A and 7B; however, unlike the implant of FIGS. 7A and 7B, the ribbon of the implant of FIG. 17 includes free ends. Additional embodiments of implants having free ends are illustrated in FIGS. 18 and 19. As shown in these implants, the pitch of the ribbon along the length of the implant can vary. Such implants can be symmetrical or asymmetrical, as desired or required.

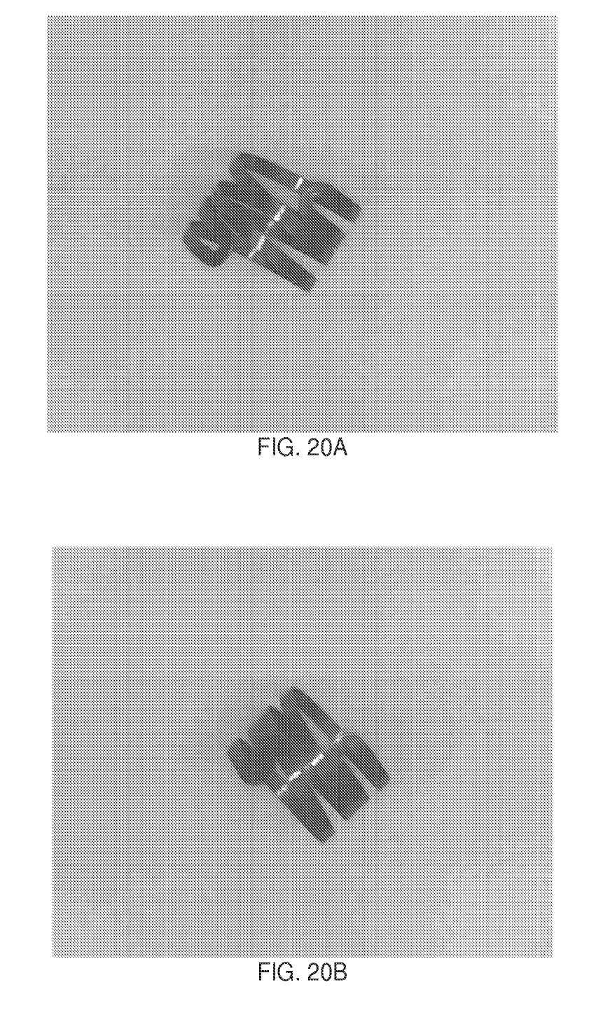

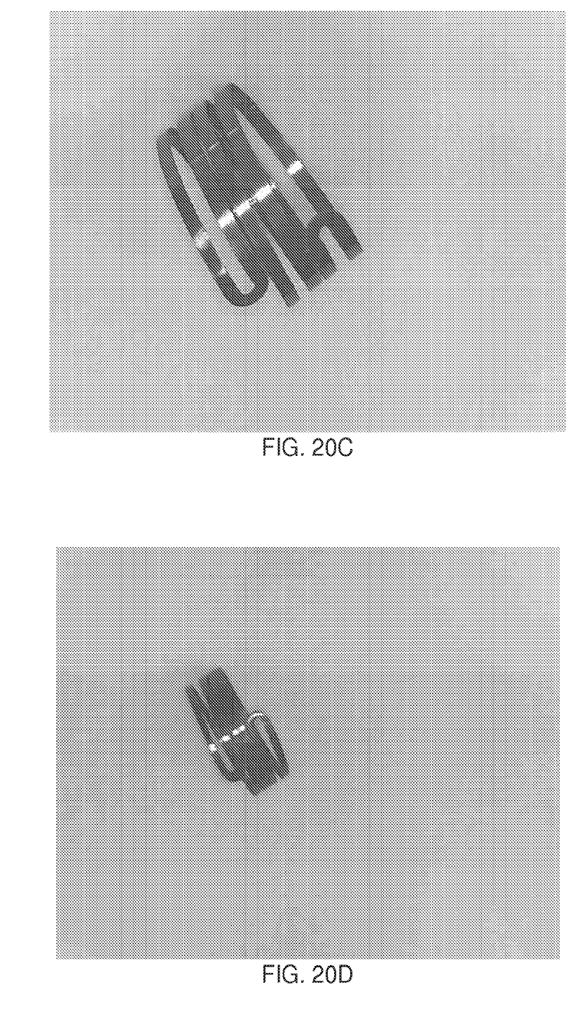

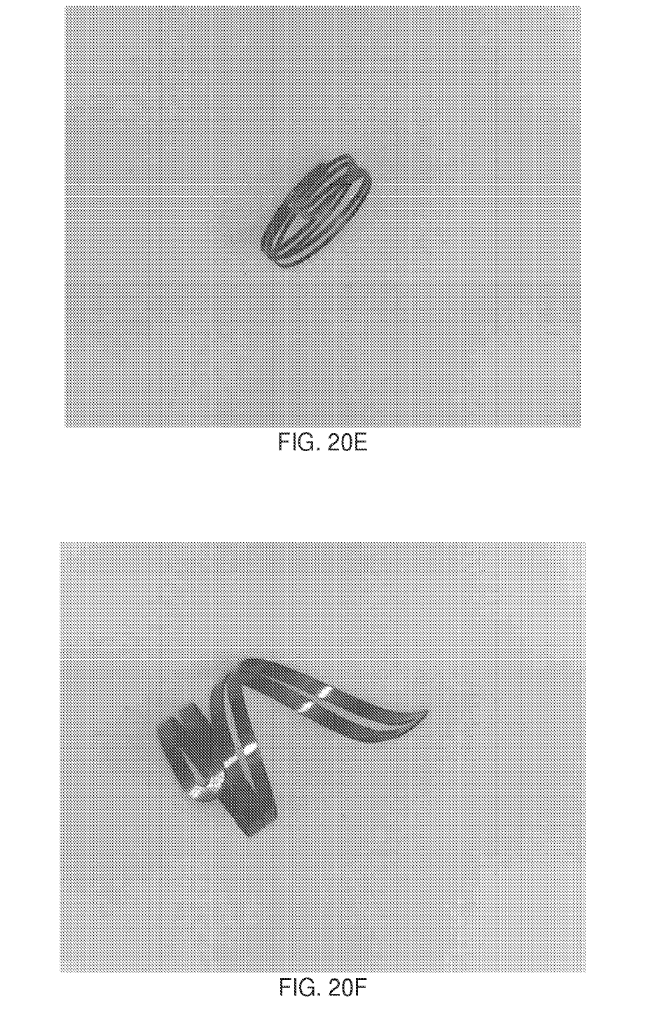

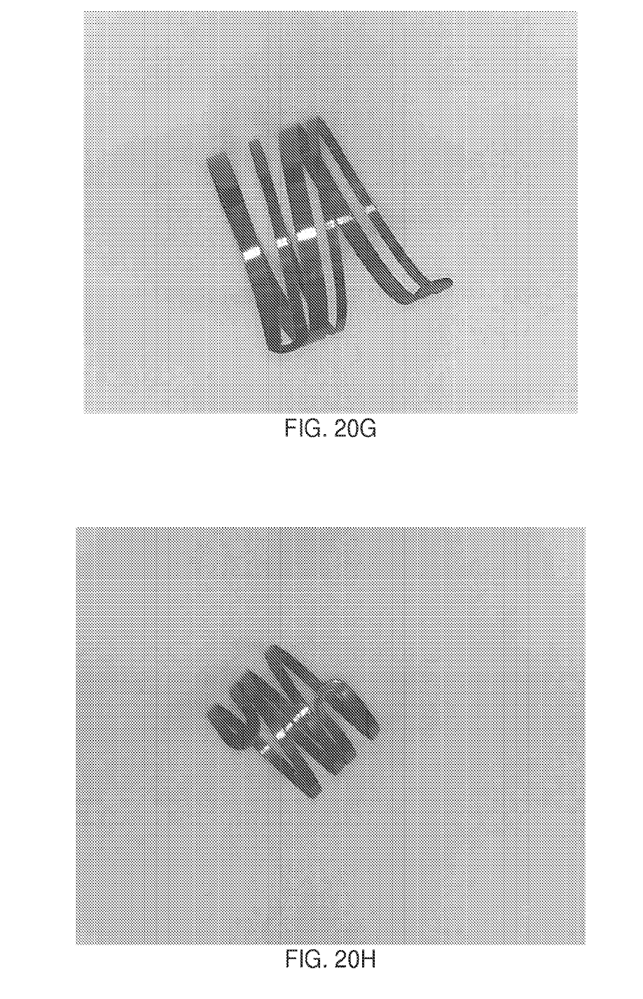

Additional embodiments of implants having a ribbon with a recess (e.g., a continuous or discontinuous central opening) are illustrated in FIGS. 20A-20H. As shown, such implants can be symmetrical or asymmetrical about a central axis. Thus, in some embodiments, the implant can include a tail or other extension along one end (e.g., see FIGS. 20F and 20G).

Implants with Enhanced End Portions

According to some embodiments, any of the implant embodiments disclosed herein and/or those illustrated and described in application explicitly incorporated by reference herein, including but not limited to U.S. patent application Ser. No. 13/830,040, can be positioned within a target vessel or other body lumen using a delivery system. Specifically, in some configurations, one or more both ends of the implant are removably secured to a catheter of the delivery system. Once the catheter and implant have been advanced to a target anatomical location (e.g., at least partially percutaneously via the vasculature and/or other body lumen of the subject), one or both ends of the implant that are removably secured to the delivery system can be released, thereby allowing the implant to assume its deployed configuration within the target vessel or other body lumen (e.g., pulmonary vein, renal artery, bronchi, trachea, etc.).

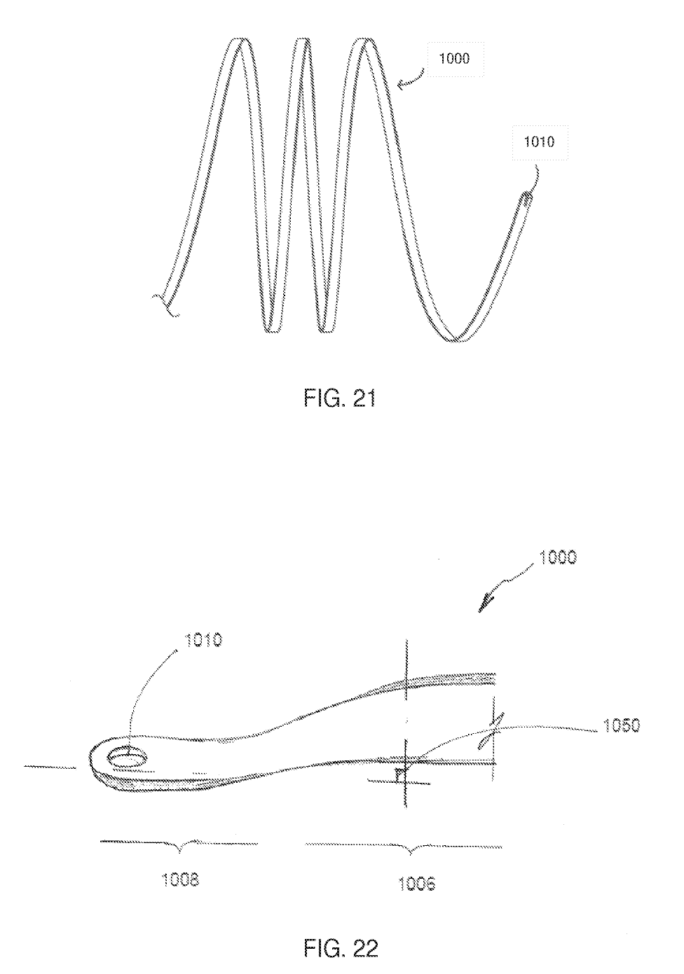

Accordingly, in order to secure an implant 1000 to a delivery system (e.g., catheter, other component of feature, etc.), one or more both ends of the implant can include a hole or other opening 1010, as illustrated in FIG. 21. However, depending on the manner in which the hole or other opening 1010 is configured to engage and at least temporarily secure to the delivery system (e.g., catheter), the hole or other opening 1010 may not be shaped, oriented and/or otherwise configured. For example, in some embodiments where the ribbon include a generally uniform shape (e.g., relative to a longitudinal axis of the implant) throughout the entire length of the implant, as shown in the configuration depicted in FIG. 21, there may exist some difficulties or other issues associated with securing the implant 1000 to the delivery system. For instance, the ribbon may become at least partially non-perpendicular with respect to the vessel or other body lumen (e.g., vein, artery, other blood vessel, airway, etc.), especially upon initial deployment (e.g., if the implant is elongated to secure to the catheter or other portion of the delivery system).

In some embodiments, a degree of canting or twisting can occur as the implant is attached to the delivery device. In some instances, it may be desirable and required to eliminate or reduce such canting during deployment. This can help eliminate or reduce any undesirable torque on the implant to help ensure it will be properly and safely deployed within the vessel or other lumen of the subject. Another potential problem associate with such designs is that the implant may undergo an undesirable or unsafe level of stress when attached to the delivery system. In some embodiments, such a stress is caused by the bend required to flex the proximal and distal ends such that the hole or other opening 1010 located along one or both ends of the implant can be engaged (e.g., by a pull wire attaching the implant to the catheter or other component of the delivery system).

Accordingly, in several embodiments, in order to reduce the stress on the implant as a result of removably attaching it to the delivery system and/or to reduce or eliminate the likelihood that an undesirable level of canting of the implant, one or more both ends of the implant's ribbon can be rotated relative to adjacent portions of the ribbon. For example, as schematically illustrated in FIG. 22, the end portion 1008 of the ribbon that includes a securement hole or opening 1010 can be rotated relative to an adjacent portion 1006 of the ribbon. In some embodiments, the end portion 1008 is rotated approximately 90 degrees (e.g., 75-105 degrees, 75-80, 80-85, 85-90, 90-95, 95-100, 100-105 degrees, values between the foregoing ranges, etc.) relative to the adjacent portion 1006. However, in other embodiments, the amount of relatively rotation between the end portion 1008 and the adjacent portion 1006 can be less than 75 degrees (e.g., 10-20, 20-30, 30-40, 40-50, 50-60, 60-65, 65-70, 70-75 degrees, values between the foregoing, less than 50 degrees, etc.) or greater than 105 degrees (e.g., 105-110, 110-115, 115-120, 120-130 degrees, values between the foregoing, greater than 130 degrees, etc.), as desired or required.

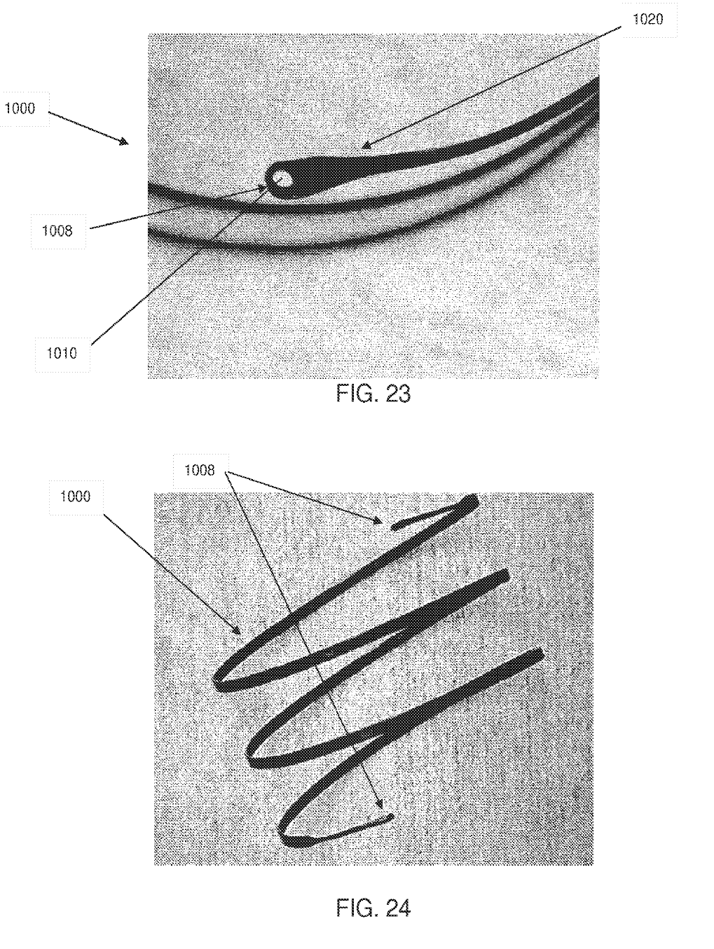

FIGS. 23 and 24 illustrate embodiments of implants 1000 comprising rotated or turned end portions 1008. As noted above, such configurations can enhance the ability of the implant to engage the pull wire and/or other portion of a delivery system while reducing or eliminating any undesirable stresses. In some embodiments, material can be removed from a neck 1020 along the end of the ribbon having an opening 1010. Alternatively, the ribbon of the implant 1000 can be initially formed or other manufactured so that it includes less material along the neck 1020 (e.g., instead of actual removing material). Regardless of how it is formed, such a reduced material portion along the neck 1020 can advantageously increase the ability of the implant end to flex, at least in part, along each end, thereby reducing or eliminating canting and the associated stresses, forces and/or moments that would otherwise be imparted on the implant 1000.

According to some embodiments, as illustrated in FIG. 23, the reduced-material section along the neck 1020 can result in a “dogbone” shape at or near one or more both ends of the implant 1000. The dogbone feature can be used in conjunction with a twisted end portion design (as discussed above) or in lieu of a rotated end portion design to help reduce canting and to alleviate stresses from the implant during delivery.

According to some embodiments, the neck comprises approximately 25% to 75% (e.g., 25-30, 30-35, 35-40, 40-45, 45-50, 50-55, 55-60, 60-65, 65-70, 70-75%, percentages within the foregoing ranges, etc.) less material (e.g., per unit length) relative to adjacent (e.g., distal and/or proximal) portions of the ribbon.

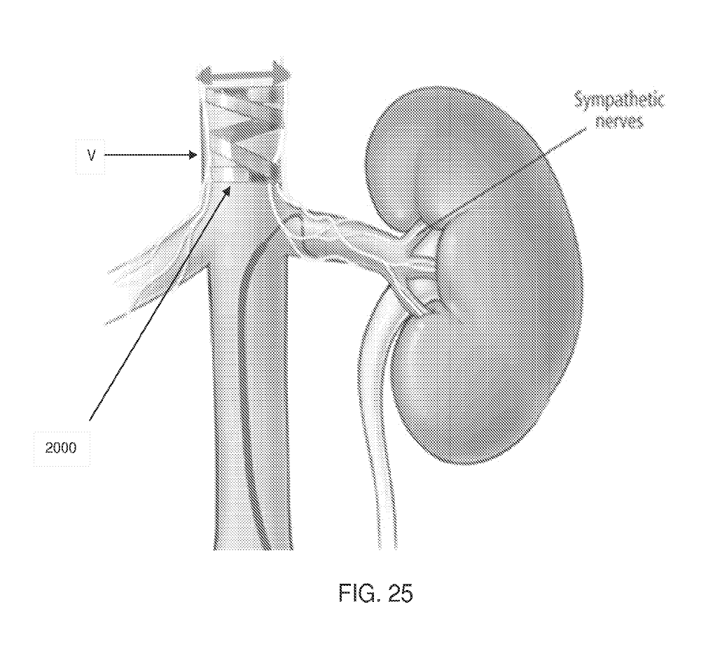

FIG. 25 illustrates one embodiment of an implant 2000 that is positioned within the aorta V adjacent the renal arteries of a subject. The implant 2000 can be configured in accordance with any of the implant embodiments disclosed and/or referenced herein. As shown, the implant 2000 can be positioned adjacent the renal arteries (e.g., but not within the renal arteries) in such a manner as to exert a pressure, force and/or a stretching effect (e.g., in the radial and/or longitudinal directions) on the aorta without penetrating the aorta. As a result of such pressure, force and/or stretching, as generally discussed in greater detail herein, the implant 2000 can disrupt the activity of the nerves traveling along the aorta the renal arteries. One advantage of positioning the implant 2000 within the aorta V is that the need to place two separate implants within the left and right renal arteries is eliminated. Thus, the necessary denervation or nerve block can be achieved by applying mechanical pressure on the main trunk nerve bundles that run along the aorta. Likewise, in other embodiments, one or more implants in accordance with the various configurations disclosed or referenced herein, can be positioned in a larger body lumen (e.g., aorta, other artery or vessel, trachea or other airway, etc.) so that the resulting electrical conduction or neural disruption caused by the implant (e.g., complete or partial signal block or interruption, denervation, etc.) can be accomplished along such body lumen. Thus, the need to treat (e.g., via separate implants) individual body lumens that are branches or divisions of the larger body lumen is advantageously eliminated or reduced. In some embodiments, such an implant and related treatment method can result in simpler, less intrusive, less dangerous and/or more efficacious results.

To assist in the description of the disclosed embodiments, words such as upward, upper, bottom, downward, lower, rear, front, vertical, horizontal, upstream, downstream have been used above to describe different embodiments and/or the accompanying figures. It will be appreciated, however, that the different embodiments, whether illustrated or not, can be located and oriented in a variety of desired positions.

Although several embodiments and examples are disclosed herein, the present application extends beyond the specifically disclosed embodiments to other alternative embodiments and/or uses of the inventions and modifications and equivalents thereof. It is also contemplated that various combinations or subcombinations of the specific features and aspects of the embodiments may be made and still fall within the scope of the inventions. Accordingly, it should be understood that various features and aspects of the disclosed embodiments can be combine with or substituted for one another in order to form varying modes of the disclosed inventions. Thus, it is intended that the scope of the present inventions herein disclosed should not be limited by the particular disclosed embodiments described above, but should be determined only by a fair reading of the claims that follow.

Various embodiments of the invention have been presented in a range format. It should be understood that the description in range format is merely for convenience and brevity and should not be construed as an inflexible limitation on the scope of the invention. The ranges disclosed herein encompass any and all overlap, sub-ranges, and combinations thereof, as well as individual numerical values within that range. For example, description of a range such as from 70 to 115 degrees should be considered to have specifically disclosed subranges such as from 70 to 80 degrees, from 70 to 100 degrees, from 70 to 110 degrees, from 80 to 100 degrees etc., as well as individual numbers within that range, for example, 70, 80, 90, 95, 100, 70.5, 90.5 and any whole and partial increments therebetween. Language such as “up to,” “at least,” “greater than,” “less than,” “between,” and the like includes the number recited. Numbers proceeded by a term such as “about” or “approximately” include the recited numbers. For example, “about 4 mm” includes “4 mm”.

1. An implant configured for placement within a target vessel or other body lumen of a subject, comprising:
a ribbon having a generally rectangular cross-sectional shape, the ribbon comprising at least a partially helical shape;
wherein the ribbon comprises a smooth outer surface with no penetrating elements or features;
wherein the implant is configured to be moved between a radially collapsed configuration and a radially deployed configuration, wherein the implant is in the radially collapsed configuration during intraluminal delivery of the implant to the target vessel or other body lumen;
wherein, when in the radially deployed configuration, the implant radially expands to contact and exert pressure along adjacent tissue of the target vessel or other body lumen without penetrating said adjacent tissue;
wherein the pressure exerted by the deployed implant at least partially blocks electrical signals along the target vessel or other body lumen; and
wherein the ribbon comprises at least one opening that extends at least partially along a length of the implant.

2. The implant of claim 1, wherein the at least one opening of the ribbon extends from or near a first end of the ribbon to a second end of the ribbon.

3. The implant of claim 1, wherein the at least one opening of the ribbon is continuous.

4. The implant of claim 1, wherein the at least one opening of the ribbon is generally discontinuous.

5. The implant of claim 4, wherein the at least one opening comprises at least one strut or rib extending across at least a portion of the at least one opening.

6. The implant of claim 1, wherein a pitch of adjacent windings of the ribbon is generally constant along an entire length of the implant.

7. The implant of claim 1, wherein a pitch of adjacent windings of the ribbon varies along at least a portion of a length of the implant.

8. The implant of claim 7, wherein the implant comprises a tail or extension along a first end of the implant, wherein a pitch of adjacent windings is greater along the first end than a pitch of adjacent windings along a second end of the implant.

9. The implant of claim 1, wherein a space exists between adjacent windings of the ribbon.

10. The implant of claim 1, wherein at least two adjacent windings of the ribbon including a space.

11. The implant of claim 1, wherein the ribbon is symmetrical along a longitudinal axis of the implant.

12. The implant of claim 1, wherein the ribbon is asymmetrical along a longitudinal axis of the implant.

13. The implant of claim 1, wherein the ribbon comprises a width of 3 mm to 5 mm.

14. The implant of claim 13, wherein the width of the ribbon is approximately 4 mm.

15. The implant of claim 1, wherein the ribbon comprises a shape memory material.

16. The implant of claim 1, wherein at least one end of the ribbon comprises a hole for securing to a delivery system, wherein the at least one end of the ribbon comprising the hole is rotated related to an immediately adjacent portion of the ribbon to reduce undesirable torque or stresses on the implant during or after implanting.

17. The implant of claim 16, wherein the at least one end of the ribbon is rotated about 90 degrees relative to the immediately adjacent portion of the ribbon.

18. The implant of claim 1, wherein at least one end of the ribbon comprises a hole for securing to a delivery system, wherein the at least one end of the ribbon comprising the hole comprising a reduced-material neck region.

19. The implant of claim 1, wherein, when in the deployed configuration, the implant is configured to stretch, at least partially, the adjacent vessel or other body lumen.

20. The implant of claim 19, wherein the implant is configured to stretch the adjacent vessel or other body lumen in the radial and longitudinal directions.

21. An implant configured for placement within a target vessel or other body lumen of a subject, comprising:
a ribbon having a generally rectangular cross-sectional shape, the ribbon comprising at least a partially helical shape;
wherein the ribbon comprises a smooth outer surface with no penetrating elements or features;
wherein the implant is configured to be moved between a radially collapsed configuration and a radially deployed configuration, wherein the implant is in the radially collapsed configuration during intraluminal delivery of the implant to the target vessel or other body lumen;
wherein, when in the radially deployed configuration, the implant radially expands to contact and exert pressure along adjacent tissue of the target vessel or other body lumen without penetrating said adjacent tissue;
wherein the pressure exerted by the deployed implant at least partially blocks electrical signals along the target vessel or other body lumen;
wherein the ribbon comprises a tail or extension along a first end of the implant, wherein a pitch of adjacent windings of the ribbon along the tail or extension is greater along the first end than a pitch along a second end of the implant such that the implant is generally asymmetrical.

22. The implant of claim 21, wherein the ribbon comprises at least one opening that extends at least partially along a length of the implant.

23. The implant of claim 22, wherein the at least one opening of the ribbon extends from or near the first end of the ribbon to the second end of the ribbon.

24. The implant of claim 21 or 22, wherein the at least one opening of the ribbon is continuous.

25. The implant of claim 21 or 22, wherein the at least one opening of the ribbon is generally discontinuous.

26. The implant of claim 25, wherein the at least one opening comprises at least one strut or rib extending across at least a portion of the at least one opening.

27. The implant of claim 21, wherein at least two adjacent windings of the ribbon including a space.

28. The implant of claim 21, wherein the ribbon comprises a width of 3 mm to 5 mm.

29. The implant of claim 28, wherein the width of the ribbon is approximately 4 mm.

30. The implant of claim 21, wherein the ribbon comprises a shape memory material.

31. The implant of claim 21, wherein at least one end of the ribbon comprises a hole for securing to a delivery system, wherein the at least one end of the ribbon comprising the hole is rotated related to an immediately adjacent portion of the ribbon to reduce undesirable torque or stresses on the implant during or after implanting.

32. The implant of claim 31, wherein the at least one end of the ribbon is rotated about 90 degrees relative to the immediately adjacent portion of the ribbon.

33. The implant of claim 21, wherein at least one end of the ribbon comprises a hole for securing to a delivery system, wherein the at least one end of the ribbon comprising the hole comprising a reduced-material neck region.

34. The implant of claim 21, wherein, when in the deployed configuration, the implant is configured to stretch, at least partially, the adjacent vessel or other body lumen.

35. The implant of claim 34, wherein the implant is configured to stretch the adjacent vessel or other body lumen in the radial and longitudinal directions.

36. An implant configured for placement within a target vessel or other body lumen of a subject, comprising:
a ribbon having a generally rectangular cross-sectional shape, the ribbon comprising a generally flat V-shape prior to being formed into cylindrical shape;
wherein the ribbon comprises a smooth outer surface with no penetrating elements or features;
wherein the implant is configured to be moved between a radially collapsed configuration and a radially deployed configuration, wherein the implant is in the radially collapsed configuration during intraluminal delivery of the implant to the target vessel or other body lumen;
wherein, when in the radially deployed configuration, the implant radially expands to contact and exert pressure along adjacent tissue of the target vessel or other body lumen without penetrating said adjacent tissue;
wherein the pressure exerted by the deployed implant at least partially blocks electrical signals along the target vessel or other body lumen; and
wherein the ribbon is generally symmetrical about a longitudinal axis and having two free ends along either side of the implant.

37. The implant of claim 36, wherein the ribbon comprises at least one opening that extends at least partially along a length of the implant.

38. The implant of claim 37, wherein the at least one opening of the ribbon extends from or near the first end of the ribbon to the second end of the ribbon.

39. The implant of claim 37, wherein the at least one opening of the ribbon is continuous.

40. The implant of claim 37, wherein the at least one opening of the ribbon is generally discontinuous.

41. The implant of claim 40, wherein the at least one opening comprises at least one strut or rib extending across at least a portion of the at least one opening.

42. The implant of claim 36, wherein the ribbon comprises a width of 3 mm to 5 mm.

43. The implant of claim 42, wherein the width of the ribbon is approximately 4 mm.

44. The implant of claim 36, wherein the ribbon comprises a shape memory material.

45. The implant of claim 36, wherein at least one end of the ribbon comprises a hole for securing to a delivery system, wherein the at least one end of the ribbon comprising the hole is rotated related to an immediately adjacent portion of the ribbon to reduce undesirable torque or stresses on the implant during or after implanting.

46. The implant of claim 45, wherein the at least one end of the ribbon is rotated about 90 degrees relative to the immediately adjacent portion of the ribbon.

47. The implant of claim 36, wherein at least one end of the ribbon comprises a hole for securing to a delivery system, wherein the at least one end of the ribbon comprising the hole comprising a reduced-material neck region.

48. The implant of claim 36, wherein, when in the deployed configuration, the implant is configured to stretch, at least partially, the adjacent vessel or other body lumen.

49. The implant of claim 48, wherein the implant is configured to stretch the adjacent vessel or other body lumen in the radial and longitudinal directions.

50. The implant of claim 1, wherein the opening comprises a slot.

51. The implant of claim 1, wherein the opening comprises at least one of a window, a slit, a recess and a pore.
